(19) United States
(12) Patent Application Publication
(10) Pub. No.: US 2023/0002502 A1
(43) Pub. Date: Jan. 5, 2023

(54) METHODS AND PRODUCTS FOR REDUCING SIDE EFFECTS ASSOCIATED WITH USE OF IMMUNE AGONIST ANTIBODIES

(71) Applicants: South Australian Health and Medical Research Institute Limited, Adelaide (AU); The Flinders University of South Australia, Bedford Park (AU)

(72) Inventors: David John Lynn, Bellevue Heights (AU); Stephen James Blake, Netherby (AU)

(73) Assignees: South Australian Health and Medical Research Institute Limited, Adelaide (AU); The Flinders University of South Australia, Bedford Park (AU)

(21) Appl. No.: 17/779,908

(22) PCT Filed: Nov. 26, 2020
(86) PCT No.: PCT/AU2020/051278

§ 371 Date: May 25, 2022

(30) Foreign Application Priority Data
Nov. 26, 2019 (AU) 2019904458

(51) Int. Cl. C07K 16/28; C07K 16/24

(52) U.S. Cl. CPC C07K 16/2878; C07K 16/241; C07K 16/2818; C07K 16/2827; C07K 2317/75

(57) ABSTRACT

The present disclosure relates to methods and products for reducing side effects associated with immunotherapy using immune agonist antibodies. In certain embodiments, the present disclosure provides a method of reducing one or more side effects associated with immunotherapy using an immune agonist antibody alone, or in combination with an immune checkpoint inhibitor, in a subject suffering from a cancer, the method comprising modifying the gut microbiota in the subject and thereby reducing the one or more side effects associated with the immunotherapy in the subject.

### PRIORITY CLAIM

This application claims priority to Australian Provisional Patent Application 2019904458 filed on 26 Nov. 2020, the contents of which are hereby incorporated by reference.

### FIELD

The present disclosure relates to methods and products for reducing side effects associated with immunotherapy using immune agonist antibodies.

### BACKGROUND

Worldwide, cancer is the second leading cause of death after heart disease. While cytotoxic chemotherapeutic agents continue to be the mainstay of cancer treatment, in recent years a variety of different immunotherapies have been developed for treating cancer. Many of these immunotherapies hold a significant promise to be able to reduce the burden of cancer disease, and in some cases treat cancers previously unresponsive to conventional treatments.

Examples of immunotherapies include therapies using monoclonal antibodies targeting specific molecules on cancer cells, cancer treatment vaccines and T-cell therapies. Immunotherapies using immune checkpoint inhibitors have also been recently developed which target specific regulators of the immune system that dampen the immune response to an immunological stimulus, and which some cancers use to protect themselves from an immunological response. Unfortunately, the efficacy of immune checkpoint inhibitor therapy is generally limited to certain types of cancers and even for cancers that are responsive, such as melanoma, the long-term survival rates are still relatively low.

It has also been recognised that targeting co-stimulatory receptors with co-agonist antibodies provides another route to treating some cancers. These immune agonists provide co-activating signals to immune cells, inducing immune cell infiltration into previously poorly infiltrated tumours, activating direct anti-tumour immunity, and increasing sensitivity to immune checkpoint inhibitor therapy.

In addition to enhancing anti-tumour immunity, immune checkpoint inhibitors and immune agonists also activate the immune system more broadly, leading to a range of immune-related adverse events in patients treated with them. Indeed, more than 80% of patients treated with an ICI will suffer from an adverse event.

In the case of immune agonists, clinical trials of these agents have been hampered by serious immunotoxicity, ranging from dose-limiting toxicity that impairs their efficacy, to serious immune-mediated side effects in some patients (˜15-30%) including cytokine release syndrome (CRS), liver damage, and even death. Further whilst clinical trials are currently being undertaken in an attempt to mitigate such side effects, many of these strategies are expected to come at the expense of anti-tumour efficacy.

Accordingly, there is a need to reduce the side effects associated with therapy using immune agonist antibodies.

### SUMMARY

The present disclosure relates to methods and products for reducing side effects associated with immunotherapy using immune agonist antibodies.

Certain embodiments of the present disclosure provide a method of reducing one or more side effects associated with immunotherapy using an immune agonist antibody in a subject suffering from a cancer, the method comprising modifying the gut microbiota in the subject and thereby reducing the one or more side effects associated with the immunotherapy in the subject.

Certain embodiments of the present disclosure provide a method of reducing one or more side effects associated with immunotherapy using an immune agonist antibody alone, or in combination with an immune checkpoint inhibitor, in a subject suffering from a cancer, the method comprising modifying the gut microbiota in the subject and thereby reducing the one or more side effects associated with the immunotherapy in the subject.

Certain embodiments of the present disclosure provide a method of reducing toxicity associated with immunotherapy using an immune agonist antibody in a subject suffering from a cancer, the method comprising modifying the gut microbiota in the subject and thereby reducing the toxicity in the subject associated with the immunotherapy.

Certain embodiments of the present disclosure provide a method of reducing one or more side effects associated with immunotherapy using an immune agonist antibody alone, or in combination with an immune checkpoint inhibitor, in a subject suffering from a cancer, the method comprising modifying the gut microbiota in the subject and thereby reducing the one or more side effects associated with the immunotherapy in the subject.

Certain embodiments of the present disclosure provide a method of preventing and/or treating one or more side effects associated with treatment of a subject with an immune agonist antibody, the method comprising modifying the gut microbiota of the subject prior to, concurrently with, and/or following treatment with the immune agonist antibody.

Certain embodiments of the present disclosure provide a method of increasing the dose of an immune agonist antibody able to be administered to a subject for immunotherapy by reducing one or more side effects associated with the administration of the immune agonist antibody, the method comprising modifying the gut microbiota in the subject and thereby increasing the dose of the immune agonist antibody able to be administered to the subject.

Certain embodiments of the present disclosure provide a method of reducing one or more side effects associated with immunotherapy using an immune agonist antibody in a subject suffering from a cancer without substantially affecting efficacy of the immunotherapy, the method comprising modifying the gut microbiota in the subject and thereby reducing the one or more side effects associated with the immunotherapy in the subject without substantially affecting the efficacy of the immunotherapy.

Certain embodiments of the present disclosure provide use of bacteria capable of modulating the toxicity of an immune agonist antibody in the preparation of a medicament for treating toxicity in a subject associated with immunotherapy using an immune agonist antibody.

Certain embodiments of the present disclosure provide use of an agent capable of modifying the gut microbiota in the preparation of a medicament for reducing one or more side effects associated with immunotherapy using an immune agonist antibody.

Certain embodiments of the present disclosure provide a method of treating a subject suffering from a cancer with an immune agonist antibody, the method comprising:
treating the subject with the immune agonist antibody; and
modifying the gut microbiota of the subject prior to, concurrently with, and/or following treatment with the immune agonist antibody.

Certain embodiments of the present disclosure provide a medicament for preventing and/or treating one or more side effects in a subject associated with treatment with an immune agonist antibody, the medicament comprising an effective amount of one or more bacteria capable of reducing one or more side effects associated with treatment with an immune agonist antibody and/or an agent for modifying the gut microbiota.

Certain embodiments of the present disclosure provide a method of treating a subject suffering from a cancer, the method comprising administering to the subject a medicament as described herein.

Certain embodiments of the present disclosure provide a combination product for treating a subject with an immune agonist antibody, the product comprising the following components.
an immune agonist antibody; and
one or more bacteria capable of modifying the gut microbiota and/or an agent capable of modifying the gut microbiota.

Certain embodiments of the present disclosure provide a method of assessing the susceptibility of a subject to one or more side effects associated with immunotherapy using an immune agonist antibody, the method comprising assessing the abundance of one or more specific species or strains of bacteria in the gut microbiota of the subject and thereby assessing the susceptibility of the subject to the one or more side effects associated with the immunotherapy using the immune agonist antibody.

Certain embodiments of the present disclosure provide a method of identifying a species or strain of bacteria capable of reducing one or more side effects associated with immunotherapy using an immune agonist antibody, the method comprising:
administering one or more types of candidate species or strains of bacteria to a subject, and
determining the ability of the candidate species or strain of bacteria to reduce one or more side effects associated with the immunotherapy,
thereby identifying the one or more types of candidate species or strains of bacteria as a species or a strain of bacteria capable of reducing one or more side effects associated with immunotherapy using an immune agonist antibody.

Certain embodiments of the present disclosure provide a method of identifying an agent capable of reducing one or more side effects associated with immunotherapy using an immune agonist antibody, the method comprising:
administering a candidate agent capable of modifying the gut microbiota to a subject; and
determining the ability of the candidate agent to reduce a side effect associated with the immunotherapy,
thereby identifying the candidate agent as an agent capable of reducing a side effect associated with immunotherapy using an immune agonist.

Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

For a better understanding of the present disclosure, and to show more clearly how the present disclosure may be carried into effect according to one or more embodiments thereof, reference will be made, by way of example, to the accompanying figures.

FIG. 1 shows antibiotic treatment reduces liver damage and cytokine release following anti-CD40 treatment without impacting its anti-tumor efficacy. (A) Overview of the experimental design. Antibiotics (ampicillin and neomycin) were administered for the duration of each experiment via the drinking water. (B) Serum ALT, (C) TNFα, (D) IL6, and (E) IFNγ levels 24 hours after control (PBS) or anti-CD40 treatment (100 μg i.p.) in antibiotic treated (ABX) and untreated (no ABX) mice. (F) H&E stained liver lateral cross-sections collected 24 hours after PBS or anti-CD40 treatment. Large sections of necrosis in no ABX+anti-CD40 treated mice are highlighted by the dotted lines. (G) Total liver histological score at 24 hours after PBS or anti-CD40 treatment. (H) Lipocalin-2 levels in feces of mice 24 hours after PBS or anti-CD40 treatment. (I) MC38 tumor growth in ABX and no ABX mice injected i.p. every 4 days with 3 doses of PBS (control) or anti-CD40 (100 μg) once tumors reached a size of ˜40-50 mm² (day 9). (J) AT3 tumor growth in ABX and no ABX mice treated with either PBS (control) or anti-CD40 (100 μg i.p.) tumors once tumors reached ˜40-50 mm² (day 17). 4 days after initial treatment mice were injected i.p. every 4 days with 3 doses of PBS (control) or anti-PD1 (200 μg i.p.). Statistical significance was determined using a Mann-Whitney test (B-I) or Kruskal-Wallis test with Dunn's post-test analysis (J). ** P≤0.01; *** P≤0.001; **** P≤0.0001. Results shown are pooled from 2 independent experiments (B-I), or from a single experiment (J).

FIG. 2 shows that antibiotics starting for only 3 or 7 days prior to anti-CD40 is sufficient to reduce its liver damage and cytokine release. Four groups of mice were inoculated with 1×106 MC38 cells s.c. and mice were either untreated (no ABX), or administered antibiotics neomycin and ampicillin (ABX) continuously via the drinking water, starting 3 or 7 days before administration of anti-CD40 (100 μg i.p.) or control (PBS). (A) Serum ALT, (B) TNFα, (C) IL6 and (D) IFNγ levels 24 hours after PBS or anti-CD40 treatment. (E) MC38 tumor growth in 3-day ABX, 7-day ABX and no ABX mice injected i.p every 4 days with 3 doses of PBS or anti-CD40 once tumors reached a size of ˜40-50 mm2 (day 10). Statistical significance was determined using a one way anova test with Dunnet's post-test analysis. * P≤0.05; ** P≤0.01; *** P≤0.001. Results shown are from a single experiment.

FIG. 3 shows antibiotic treatment reduces liver damage and cytokine release following anti-CD137 treatment with only a minor impact on its anti-tumor efficacy (A) Overview of the experimental design. Antibiotics (ampicillin and neomycin) were administered for the duration of each experiment via the drinking water. (B) Representative H&E stained liver cross-sections 11 days after initiation of anti-CD137 treatment in antibiotic treated (ABX) and untreated (no ABX) mice. Areas of immune infiltration are indicated with arrows. (C) Serum ALT levels 11 days after initiation of control (PBS) or anti-CD137 treatment (100 μg i.p.) in antibiotic treated (ABX) and untreated (no ABX) mice. (D) Total liver histological score 11 days after initiation of PBS or anti-CD137 treatment. (E) Serum IFNγ, (F) TNFα and (G) IL6 levels 11 days after initiation of PBS or anti-CD40 treatment. (H) MC38 tumor growth in ABX and no ABX treated mice injected i.p. every 4 days with 3 doses of PBS or anti-CD137 (100 μg) once tumors reached a size of ˜40-50 mm² (day 10). (I) Tumor rejection of mice treated with PBS or anti-CD137. (J) Percentages of tumor infiltrating CD8⁺ T cells (left), FoxP3⁺ regulatory T cells (middle) and Ki67⁺ CD8⁺ T cells (right) 11 days after initiation of PBS or anti-CD137 treatment. Statistical significance was determined by Mann-Whitney test. * P≤0.05; ** P≤0.01; *** P≤0.001; **** P≤0.0001. Results shown are pooled from 2 independent experiments (C-G, I,J) or from a single experiment (H).

FIG. 4 shows that germ-free mice have significantly reduced anti-CD40 and anti-CD137 induced immunotoxicity. This immunotoxicity is restored when germ-free mice recolonized with a commensal microbiota by fecal microbiota transplant (FMT) from SOPF mice. (A) Overview of the experimental design. (B) 16S rRNA gene sequencing was used to profile the composition of the microbiota in GF+FMT and SOPF mice. GF+FMT and SOPF mice had a similar diversity and composition of the gut microbiota. (C) Serum ALT and (D) Liver histological score at 24 hours after control (PBS i.p.) or anti-CD40 treatment (100 μg i.p.) in GF or GF+FMT mice. (E) TNFα, (F) IL6 levels in serum collected 24 hours after PBS or anti-CD40 treatment. GF mice were alternatively re-colonized with monocultures of Enterobacter cloacae, Clostridium scindens or Akkermansia muciniphila. (G) ALT (H) TNFα, (I) IL6 levels in serum collected 24 hours after PBS (Control) or anti-CD40 (100 μg i.p.) treated SOPF mice, or monoculture recolonized GF mice. (J) Total liver histological score, (K) TNFα or (L) IL6 levels in serum collected 11 days after treatment initiation with anti-CD137 (100 μg i.p.) or PBS (control) in GF and GF+FMT mice. Significance was determined using a Wilcoxon Rank Sum test (B) or Mann-Whitney test (C-L). * P≤0.05; ** P≤0.01; *** P≤0.001; **** P≤0.0001. Results shown are from a single experiment (D, G-L) or pooled from 2 independent experiment (C, E-F).

FIG. 5 shows antibiotic treatment reduces the inflammatory myeloid and lymphocyte cell liver infiltration that is induced by anti-CD40 or anti-CD137. (A) representative figure showing neutrophils (CD11b+Ly6G+) and monocytes/macrophages (CD11b+Ly6G−) and monocyte/macrophage expression of activation markers CD86 and CD80 in the liver of untreated or antibiotic treated (ABX) mice 24 hours after treatment with control (PBS i.p.) or anti-CD40 (100 μg i.p.).number/frequency of (B) monocytes/macrophages, (C) conventional dendritic cells (cDCs; CD11c+MHCII+), (D) monocytes/macrophages expressing CD80, (E) neutrophils, (F) natural killer cells (NK; NK1.1+CD3−), (G) and CD8⁺ T-cells (CD8+TCRβ+) as determined by flow cytometry analysis of livers collected 24 hours after control (PBS i.p) or anti-CD40 treatment (100 μg i.p.) in ABX and no ABX mice. Number of (H) monocytes/macrophages, (I) cDCs, (J) and CD8⁺ T-cells as determined by flow cytometry analysis of livers collected 11 days after treatment initiation with anti-CD137 (100 μg i.p.) or PBS in ABX and no ABX mice. Statistical significance was determined using a Mann-Whitney test. * P≤0.05; ** P≤0.01; *** P≤0.001; **** P≤0.0001; not-significant (N.S.). Results shown are pooled from 2 independent experiments.

FIG. 6 shows antibiotic treatment and anti-CD40 modulate liver gene expression. RNA-Seq was used to profile gene expression in liver samples collected from mice 24 hours after treatment with control (PBS) or anti-CD40 (100 μg i.p.) either untreated (No ABX) or treated with antibiotics (ABX). (A) Multidimensional scaling (MDS) analysis of RNA-Seq data. (B) Heatmap showing differentially expressed genes (by FDR<0.05)) in 4 GO terms of interest. (C-D) Top GO terms enriched among differentially expressed gene sets (hypergeometric test, FDR<0.05) between Control and Anti-CD40 (C), and No ABX and ABX during anti-CD40 treatment (D). (F-H) Tuckey style boxplots showing library size normalized expression (log 2 count per million) of Tlr4 (F), Ccl3 (E), Ccl4 (G), and Il1b (H). (I) Line plot showing individual gene expression (log 2 library size normalized) across each of the 4 treatment combinations for inflammatory response genes (GO:0006954) which are decreased (FDR<0.05) in response to ABX. Intensity represents Z score of log 2 library size normalized counts. Significance determined in EdgeR using the genewise negative binomial generalized linear model (implemented within the EdgeR library, glmQLFTest function) and adjusted for multiple tests with the false discovery rate. * P≤0.05; ** P≤0.01; *** P≤0.001.

FIG. 7 shows that the cytokine release syndrome induced by anti-CD40 treatment is partially dependent on the MyD88 pathway, but not NOD2 or TLR4 pathways. (A) Serum ALT, TNFα, and IL6 levels 24 hours after treatment with control (PBS) or anti-CD40 (100 μg i.p.) in littermate Myd88^{+/+} (Wildtype) or Myd88^{-/-} mice. (B) Serum ALT, TNFα, and IL6 levels 24 hours after treatment with control (PBS) or anti-CD40 (100 μg i.p.) in littermate Tlr4^{+/+} (Wildtype) or Tlr4^{-/-} mice. (C) Serum ALT, TNFα, and IL6 levels 24 hours after treatment with control (PBS) or anti-CD40 treatment (100 μg i.p.) in co-housed Nod2^{+/+} (Wildtype) or Nod2^{-/-} mice. MC38 tumor growth in (D) littermate Myd88^{+/+} (Wildtype) or Myd88^{-/-} mice, (E) littermate Tlr4^{-/-} (Wildtype) or Tlr4^{-/-} mice, (F) in co-housed Nod2^{+/+} (Wildtype) or Nod2^{-/-} mice that were injected i.p. every 4 days with 3 doses of PBS (control) or anti-CD40 (100 μg) once tumors reached a size of ˜40-50 mm² (treatment initiation indicated with an arrow) Statistical significance was determined using a Mann-Whitney test. * P≤0.05; ** P≤0.01; *** P≤0.001; **** P≤0.0001; Not significant (N.S.). Results shown are pooled from 2 independent experiments (A,B) or from a single experiment (C-F).

FIG. 8 shows that the gut microbiota modulates liver bile acids without impacting anti-CD40 induced liver damage (A) Quantification of primary bile acid TbMCA (tauro beta muricholic acid) and secondary bile acids TωMCA (tauro omega muricholic acid) and TDCA (tauro deoxycholic acid) in the livers of germ free (GF), or GF mice recolonized with an FMT from SOPF donors (GF+FMT) 24 hours after treatment with control (PBS) or anti-CD40 (100 μg i.p.). (B) Spearman correlation analysis of serum cytokines levels, liver bile acid concentrations, liver immune cell population frequencies, and ALT levels measured 24 hours after treatment with PBS or anti-CD40 (100 μg i.p.) in GF and GF+FMT mice, (C) or in antibiotic treated (ABX) and untreated (no ABX) mice. Node size is proportional to control SOPF values, rescaled as 1-10. Edge width and intensity is proportional to strength of correlation (minimum cor=0.3, p<0.05). Correlations generated across all samples from all groups. (D) Serum ALT, (E) TNFα, (F) IL6 (G) IFNγ levels 24 hours after treatment with control (PBS) or anti-CD40 (100 μg i.p.) in untreated (no ABX) mice or mice treated continually with a 2% cholestyramine diet (CHOL), starting 7 days before anti-CD40 administration. Statistical significance was determined using a Mann-Whitney test. * P≤0.05; ** P≤0.01; Not significant (N.S.). Results shown are from a single experiment.

FIG. 9 shows that liver macrophages and TNFα are critical mediators of anti-CD40 induced toxicity. (A) Serum ALT, TNFα, IL6, and IFNγ levels 24 hours after control (PBS) or anti-CD40 treatment (100 μg i.p.) in mice treated with control PBS loaded liposomes (Lip-PBS) or clodronate loaded liposomes (Lip-Clod) 24 hours prior to anti-CD40. (B) Serum ALT, TNFα, IL6, and IFNγ levels 24 hours after control or anti-CD40 treatment. Indicated groups were also treated concurrently with anti-TNF (200 μg i.p.) or anti-IL1β (200 μg i.p.) blocking antibodies. (C) number of monocytes/macrophages (CD11b⁺Ly6G⁻), (D) monocyte/macrophage expression of activation marker CD80, (E) number of conventional dendritic cells (cDCs; CD11c⁺MHCII⁺), (F) cDC expression of CD80, (G) number of neutrophils (CD11b⁺Ly6G⁺), (H) number of natural killer cells (NK; NK1.1⁺TCRβ⁻), (G) number of CD8+ T-cells (CD8⁺ TCRβ⁻) and (J) number of natural killer T-cell like cells (NKT-like, NK1.1⁺TCRβ⁺) was determined 24 hours after treatment with control or anti-CD40, with indicated groups also receiving concurrent anti-TNF or anti-IL1β antibodies. Statistical significance was determined using a Mann-Whitney test. * P≤0.05; ** P≤0.01; *** P≤0.001; **** P≤0.0001; not-significant (N.S.). Results shown are pooled from 2 independent experiments (A) or from a single experiment (B-J).

FIG. 10 shows that anti-OX40 induces mild liver inflammation that is also reduced by antibiotic treatment. (A) 5-week old, male C57BL/6 mice were treated with an antibiotic cocktail of ampicillin and neomycin (ABX) in water which they had access to ad libitum. 7 days later (D0), 4×105 MC38 tumor cells were subcutaneously (s.c.) injected into the right flank of mice. Mice were treated i.p. with 100 ug of αOX40 or PBS (control) 3 times in 4 day intervals. Mice were culled D11 and livers were harvested for histological analysis. n=10 for each group of mice. (B) Livers were harvested at D10 preserved in formalin. Liver sections were then embedded in paraffin and sectioned for haematoxylin & eosin staining. Stained samples were viewed under 20× magnification. A representative image for each group is shown above. (C) Histological scoring for total inflammation, portal inflammation, lobular inflammation and necrosis for liver sections from each group. A Mann-Whitney test was used to determine statistical significance (N.S.=Not significant, * P≤0.05, ** P≤0.01).

### DETAILED DESCRIPTION

The present disclosure relates to methods and products for reducing side effects associated with therapy using immune agonist antibodies.

The present disclosure also relates to methods and products for increasing the effective dose of an immune agonist antibody able to be administered to a subject, methods and products for assessing the susceptibility of a subject to one or more side effects associated with treatment with an immune agonist antibody, the identification of bacteria capable of reducing one or more side effects associated with use of an immune agonist antibody, and the identification of agents capable of reducing side effects associated with use of an immune agonist antibody.

The present disclosure is based, at least in part, on the demonstration that the immunotoxicity associated with immune agonists is dependent on the gut microbiota. Germ-free or antibiotic-treated tumour-bearing mice have significantly reduced induced immunotoxicity to the immune agonists, and cytokine storm, and severe liver damage is almost completely abolished. The studies indicate that microbiota-targeted interventions substantially reduce the immunotoxicity associated with immune agonists, overcoming a critical roadblock to their clinical application.

As a consequence, reducing immunotoxicity associated with immune agonist therapy provides a number of benefits including reducing cancer patient morbidity, reducing costs to health care system by having fewer patients with serious side effects and more patients who are responsive to the costly treatments, and enhancing cancer immunotherapy by enabling higher doses of therapeutic agents to be administered due to lower risks of serious side effects.

Certain embodiments of the present disclosure provide a method of reducing one or more side effects associated with therapy using an immune agonist antibody in a subject suffering from a cancer.

In certain embodiments, the present disclosure provides a method of reducing one or more side effects associated with immunotherapy using an immune agonist antibody in a subject suffering from a cancer, the method comprising modifying the gut microbiota in the subject and thereby reducing the one or more side effects associated with the immunotherapy in the subject.

The term “gut microbiota” as used herein refers to the population of microorganisms present in the gastrointestinal tract. The microorganisms include the bacteria, archaea, viruses and eukarya present in the gastrointestinal tract.

In this regard, the modifying of the gut microbiota of the present disclosure refers to an effective treatment that changes the abundance, characteristics and/or function of one or more microorganisms in the gastrointestinal tract, and includes treatments that reduce the abundance of one or more species/strains of microorganisms, treatments that introduce one or more new species/strains of microorganisms to the gastrointestinal tract, treatments that modify the microorganisms, treatments that alter the production of one or more molecular species by the microorganisms, and/or treatments that alter an interaction between the microorganisms and/or the host.

In certain embodiments, the subject is a human subject. Veterinary applications of the present disclosure are also contemplated.

In certain embodiments, the method reduces one or more side effects associated with immunotherapy using an immune agonist antibody alone, or in combination with an immune checkpoint inhibitor. Immunotherapies using immune agonist antibodies alone, or in combination with immune checkpoint inhibitors, are known in the art for example as described in Vonderheide (2018) Cancer Cell. 33(4): 563-569 and in “Immune Checkpoint Inhibitors in Cancer” (2018) Ito F. and Ernstoff M. S, Elsevier, ISBN: 978-0-323-54948-6.

In certain embodiments, the one or more side effects comprise one or more of liver toxicity, increased cytokine expression, cytokine release syndrome, colitis, dermatitis, elevated levels of liver enzymes in serum, liver toxicity, liver necrosis, a haematological toxicity, and death. Other types of side effects are contemplated. Determination of the presence and/or extent of one or more side effects may be made by a suitably qualified medical practitioner, in combination with the appropriate clinical tests as required.

Examples of immune agonist antibodies are described for example in Mayes, P. A., Hance, K. W. & Hoos, A. (2018) Nat Rev Drug Discov 17, 509-527. Methods using immune agonists for treatment are described, for example, in Segal et al. (2019) Clinical Cancer Res. 23(8): 1929 and Hassan et al. (2014) Immunopharmacology and Immunotoxicology 36(2):96-104.

Examples of immune agonist antibodies are provided in Table 1.

TABLE 1
Immune Agonists

| Target | Agonist | Antibody Isotype |
|---|---|---|
| CD27 | Varlilumab (CDX-1127) | IgG1 |
| CD40 | CDX-1140 | IgG2 |
| | SEA-CD40 | Non-fucosylated IgG1 |
| | RO7009789 | IgG2 |
| | JNJ-64457107 (ADC1013) | IgG1 |
| | APX-005M | IgG1 |
| | Chi Lob 7/4 | Mouse/human |
| | | chimaera IgG1 |
| GITR | TRX-518 | Aglycosyl IgG1 |
| | MK-4166 | IgG1 |
| | MK-1248 | IgG4 |
| | GWN-323 | IgG1 |
| | INCAGN01876 | IgG1 |
| | BMS-986156 | IgG1 |
| | AMG-228 | IgG1 |
| OX40 | Tavolimab (MEDI0562) | IgG1 |
| | PF-04518600 | IgG2 |
| | BMS-986178 | IgG1 |
| | MOXR-0916 | IgG1 |
| | GSK-3174998 | IgG1 |
| | INCAGN01949 | IgG1 |
| 4-1BB (CD137) | Utomilumab (PF-05082566) | IgG2 |
| | Urelumab (BMS-663513) | IgG4 |
| ICOS | GSK-33 59609 | IgG4 |
| | JTX-2011 | IgG1 |
| CD28 | Theralizumab (TAB-08) | IgG4 |

Immune agonist antibodies are available and/or may be produced by a method known in the art. Methods for treating a cancer with an immune agonist antibody are known in the art.

In certain embodiments, the immune agonist antibody comprises one or more of an anti-CD137 antibody, anti-CD40 antibody, anti-CD28 antibody, anti-GITR antibody and anti-OX40 antibody. The aforementioned immune agonist antibodies are known in the art and described, for example, in Mayes et al (2018) Nature Reviews Drug Discovery 17(7): 509-527.

The term “antibody” as used herein refers to an immunoglobulin molecule with the ability to bind an antigenic region of another molecule, and includes monoclonal antibodies, polyclonal antibodies, multivalent antibodies, chimeric antibodies, multispecific antibodies, diabodies, and a part or fragment of an immunoglobulin molecule or combinations thereof that have the ability to bind to the antigenic region of another molecule with the desired affinity including a Fab, Fab′, F(ab′)₂, Fv, a single-chain antibody (scFv) or a polypeptide that contains at least a portion of an immunoglobulin (or a variant of an immunoglobulin) that is sufficient to confer specific antigen binding, such as a molecule including one or more CDRs.

In certain embodiments, the subject is suffering from a cancer as described herein.

Examples of cancers that may be suitable for treatment with an immune agonist antibody include solid tumour cancers, melanoma, non-small cell lung cancer, breast cancer, gastric cancer, renal cell carcinoma, ovarian cancer, cholangiocarcinoma, bladder urothelial carcinoma, pancreatic adenocarcinoma, colorectal cancer, esophageal cancer, hepatic cancer, head and neck cancer, primary peritoneal cancer, fallopian tube cancer, other solid tumours, diffuse large b-cell lymphoma, mantle cell lymphoma, indolent b-cell lymphomas, and non-hodgkin's lymphoma. Other types of cancers are contemplated.

In certain embodiments, the subject is suffering from a cancer as provided in Table 2 and which may be treated with a suitable immune agonist antibody as provided.

TABLE 2

| Target | Agonist | Cancer |
|---|---|---|
| CD27 | Varlilumab (CDX-1127) | Solid tumours |
| CD40 | CDX-1140 | Lymphoma |
| | SEA-CD40 | Solid tumours, |
| | | lymphomas |
| | RO7009789 | Solid tumours, |
| | | Pancreatic cancer |
| | JNJ-64457107 (ADC1013) | CRC, HNSCC, urothelial |
| | | carcinoma, and |
| | | melanoma |
| | APX-005M | NSCLC, Melanoma, |
| | | urothelial cancer, |
| | | HNSCC, esophageal |
| | | and gastroesophageal |
| | | cancers, |
| GITR | TRX-518 | Solid tumours |
| | MK-4166 | Solid tumours |
| | MK-1248 | Solid tumours |
| | GWN-323 | Solid tumours, |
| | | lymphomas |
| | INCAGN01876 | Solid tumours |
| | BMS-986156 | Solid tumours |
| OX40 | Tavolimab (MEDI0562) | Solid tumours |
| | PF-04518600 | Melanoma, NSCLC |
| | BMS-986178 | Solid tumours |
| | MOXR-0916 | Solid tumours |
| | GSK-3174998 | Solid tumours |
| | INCAGN01949 | Solid tumours |
| 4-1BB | Utomilumab (PF-05082566) | Solid tumours |
| (CD137) | | |
| | Urelumab (BMS-663513) | Solid tumours, NHL |
| ICOS | GSK-3359609 | Solid tumours |
| | JTX-2011 | Solid tumours |
| CD28 | Theralizumab (TAB-08) | CLL |

In certain embodiments, the immunotherapy further comprises use of an immune checkpoint inhibitor, or with other therapies. Methods for treating a subject with an immune agonist antibody alone, in combination with an immune checkpoint inhibitor, or with other therapies are known in the art.

In certain embodiments, the immune checkpoint inhibitor comprises one or more of an anti-PD1 antibody, an anti-CTLA4 antibody, an anti-PDL1 antibody, an anti-TIGIT antibody, an anti-Lag3 antibody and an anti-Tim3 antibody. Other immune checkpoint inhibitors are contemplated.

Examples of combination treatments include varlilumab (CD27 agonist) with nivolumab (anti-PD1) for the treatment of B-cell lymphoma; varlilumab (CD27 agonist) with ipilimumab (anti-CTLA4) for the treatment of melanoma, varlilumab (CD27 agonist) with atezolizumab (anti-PD1) for treatment of solid tumors; APX005M (CD40 agonist) with pembrolizumab (anti-PD1) for treatment of melanoma; APX005M (CD40 agonist) with nivolumab (anti-PD1) for treatment of melanoma NSCLC; RO7009789 (CD40 agonist) with atezolizumab (anti-PD1) for the treatment of solid tumors, utomilumab (CD137 agonist) with avelumab (anti-PD1) for diffuse large B-cell lymphoma; utomilumab (CD137 agonist) with avelumab (anti-PD1) for solid tumors; Urelumab (CD137 agonist) with nivolumab (anti-PD1) for the treatment of sold tumors; INCAGN01876 (GITR agonist) with ipilimumab (anti-CTLA4) for the treatment of solid tumors; GSK3359609 (ICOS agonist) with pembrolizumab (anti-PD1) for solid tumors; GSK3174998 (OX40 agonist) with pembrolizumab (anti-PD1) for the treatment of solid tumors; MEDI-0562 (OX40 agonist) with tremelimumab (anti-CLA4) for the treatment of solid tumors. The aforementioned combination treatments are as described, for example, in Cabo et al (2017) OncoImmunology 6(12):e1371896.

In certain embodiments, the modifying of the gut microbiota comprises one or more of administration to the subject of an effective amount of an antibiotic, administration to the subject of a bacteriostatic agent, administration to the subject of an effective amount of probiotic bacteria, an effective amount of a faecal transplant, administration of an effective amount of a prebiotic, an effective amount of probiotic bacteria combined with an effective amount of prebiotic, administration of an effective amount of one or more suitable foods and/or supplements, and administration of an effective amount of suitable bacteriophage.

Methods for assessing the ability of a treatment to modify the gut microbiota are known in the art, and include for example assessment of the bacterial and/or non-bacterial population at mucosal surfaces in the gastrointestinal tract or in faeces, using for example 16S rRNA sequencing, metagenome sequencing, targeted-PCR, culturing or a MALDI-Biotyper for identification.

Suitable administration routes include oral administration and/or rectal administration, which are known in the art. Formulations for delivery by the aforementioned routes are known in the art, for example in Remington's Pharmaceutical Sciences, 17th ed., Mack Publishing Company, Easton, Pa., 1985.

Effective amounts of agents to be delivered may be selected.

Oral formulations as described herein may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminium silicate, and triethanolamine. Oral formulations may also utilize standard delay or time-release formulations to alter absorption. The oral formulation may also consist of administering the active ingredient in water, food or a fruit juice, containing appropriate solubilizers or emulsifiers as needed. Oral formulations are known in the art and may be formulated by a skilled person.

For example, an oral administration using a capsule filled with the therapeutic substance may be used. Suitable dosage for administration may be selected.

Rectal delivery includes use of fecal transplants, suppositories, enemas and use of catheters. Rectal delivery methods are known in the art, and a suitable effective dosage may be selected.

In certain embodiments, the modifying of the gut microbiota comprises administration to the subject of an effective amount of an antibiotic.

Use of antibiotics for modifying the gut microbiota are known in the art, for example as described in Jernberg et al (2010) Microbiology 156, 3216-3223 Methods for use of antibiotics, and standard dosing regimes for antibiotics, are known in the art. Methods for formulating antibiotics for delivery are known in the art.

In certain embodiments, the antibiotic comprises one or more of a penicillin, a cephalosporin, a macrolide, a fluoroquinolone, a sulphonamide, a tetracycline, and a aminoglycoside. Other type of antibiotics are contemplated.

In certain embodiments, the modifying of the gut microbiota comprises administration to the subject of an effective amount of a faecal transplant. Methods and delivery forms for use in faecal transplants are known in the art, for example as described in Ooijevaar et al (2019) Annual Review of Medicine 70: 335-351.

In certain embodiments, the modifying of the gut microbiota comprises administration to the subject of an effective amount of one or more probiotic bacteria. Compositions comprising probiotic bacteria for administration are known in the art.

Examples of probiotic bacteria include Lactobacillus acidophilus, Lactobacillus bulgaricus, Lactobacillus casei, Lactobacillus gasseri, Lactobacillus plantarum, Bifidobacterium bifidum, Bifidobacterium lactis, Bifidobacterium longum, Enterococcus faecium, Saccharomyces boulardii, Akkermansia muciniphila, Collinsella aerofaciens, Enterococcus faecium, Faecalibacterium prausnitzii and Ruminococcus bromii, or a genus of any of the aforementioned species Other types of probiotic bacteria are contemplated. Probiotic bacteria may be administered live or pasteurised.

Methods and delivery forms for using probiotic bacteria are known in the art.

In certain embodiments, the modifying of the gut microbiota comprises administration to the subject of an effective amount of a prebiotic.

Examples of prebiotics include Fructans, Galacto-Oligosaccharides, Starch and Glucose-Derived Oligosaccharides, Fructooligosaccharides, human milk oligosaccharides, Non-Carbohydrate Oligosaccharides. Other type of prebiotics are contemplated. Methods for administering prebiotics are known in the art.

In certain embodiments, the modifying of the gut microbiota comprises administration to the subject of an effective amount of one or more suitable foods and/or supplements.

Examples of foods and/or supplements for modifying the gut microbiota are described in Davani-Davari et al (2019)Foods 8:92, and include fibre supplements and a Mediterranean diet. Methods for administering prebiotics are known in the art.

In certain embodiments, the modifying of the gut microbiota comprises administration to the subject of an effective amount of a suitable bacteriophage. Suitable bacteriophage able to target specific bacteria are known in the art.

The use of bacteriophage for modifying gut bacteria are known in the art, for example as described in Duan et al (2019) Nature November 13. doi: 10.1038/s41586-019-1742-x. For example, a liquid composition containing a suitable number of bacteriophage (eg 10¹⁰ to 10¹² PFUs) may be administered to the subject.

In certain embodiments, the method further comprises administering to the subject an effective amount of a TNF inhibitor, such as an anti-TNF antibody. Administration to the subject of the TNF inhibitor may occur at any one or more of prior to, concurrently with, and after modifying the gut microbiota. Examples of agents for modifying the gut microbiota areas described herein Examples of TNF inhibitors include monoclonal antibodies such as infliximab, adalimumab, certolizumab pegol, and golimumab, a receptor fusion protein such as etanercept, thalidomide and derivatives, xanthine derivatives (e.g. pentoxifylline), and bupropion. The administration may involve separate administration of a TNF inhibitor and an agent for modifying the gut microbiota, or alternatively the agents may be combined. Methods for administering TNF inhibitors are known in the art.

In certain embodiments, the method further comprises administering to the subject an effective amount of an IL-6 inhibitor. Administration to the subject of the TNF inhibitor may occur at any one or more of prior to, concurrently with, and after modifying the gut microbiota. Examples of agents for modifying the gut microbiota are as described herein. Examples of IL-6 inhibitors include monoclonal receptor antibodies such as tocilizumab or monoclonal antibodies such as siltuximab. Methods for administering IL-6 receptor inhibitors and Il-6 inhibitors are known in the art.

In certain embodiments, the method comprises assessing the susceptibility of the subject to the one or more side effects associated with the immune agonist antibody immunotherapy by assessing the abundance of one or more specific microorganisms in the gut microbiota of the subject. In certain embodiments, the method comprises assessing the susceptibility of the subject to the one or more side effects associated with the immune agonist antibody immunotherapy by assessing the abundance of one or more specific genus, species or strains of bacteria in the gut microbiota of the subject.

Methods for assessing the abundance of one or more specific species or strains of bacteria in the gut microbiota of the subject are known in the art, and include for example, 16S rRNA sequencing, metagenome sequencing, targeted-PCR, culturing or MALDI-Biotyper to identify specific bacteria, using samples collected from the gastrointestinal tract, or faeces.

In certain embodiments, the method does not substantially decrease efficacy of the immunotherapy. In certain embodiments, the method only minimally affects the efficacy of the immunotherapy. In certain embodiments, the method does not significantly abrogate the efficacy of the immunotherapy. Methods for assessing efficacy of therapy are known in the an.

In certain embodiments, the method is used to increase the dosage of an immune agonist antibody able to be administered to a subject.

Certain embodiments of the present disclosure provide a method of reducing toxicity associated with immunotherapy using an immune agonist antibody in a subject suffering from a cancer.

In certain embodiments, the present disclosure provides a method of reducing toxicity associated with immunotherapy using an immune agonist antibody in a subject suffering from a cancer, the method comprising modifying the gut microbiota in the subject and thereby reducing the toxicity in the subject associated with the immunotherapy.

In certain embodiments, the toxicity comprises one or more of liver toxicity, increased cytokine expression, cytokine release syndrome, elevated levels of liver enzymes in serum, liver necrosis, and a haematological toxicity. Other types of toxic events associated with the immunotherapy are contemplated Determination of the presence and/or extent of toxicity may be made by a suitably qualified medical practitioner.

Methods and agents for modifying the gut microbiota in the subject are as described herein.

Methods for assessing toxicity associated with immunotherapy are known in the art and as described herein. Determination of the level of toxicity may be made by a suitably qualified medical practitioner, in combination with the appropriate clinical tests as required.

Certain embodiments of the present disclosure provide a method of reducing one or more side effects associated with immunotherapy using an immune agonist antibody alone, or in combination with an immune checkpoint inhibitor.

In certain embodiments, the present disclosure provides a method of reducing one or more side effects associated with immunotherapy using an immune agonist antibody alone, or in combination with an immune checkpoint inhibitor, in a subject suffering from a cancer, the method comprising modifying the gut microbiota in the subject and thereby reducing the one or more side effects associated with the immunotherapy in the subject.

Side effects associated with immunotherapy using an immune agonist antibody alone, or in combination with an immune checkpoint inhibitor, are as described herein.

Methods and agents for modifying the gut microbiota in the subject are as described herein.

Certain embodiments of the present disclosure provide a method of preventing and/or treating one or more side effects associated with treatment of a subject with an immune agonist antibody.

In certain embodiments, the present disclosure provides a method of preventing and/or treating one or more side effects associated with treatment of a subject with an immune agonist antibody, the method comprising modifying the gut microbiota of the subject prior to, concurrently with, and/or following treatment with the immune agonist antibody.

Side effects associated with immunotherapy using an immune agonist antibody alone, or in combination with an immune checkpoint inhibitor, are as described herein.

Methods for modifying the gut microbiota in the subject areas described herein.

In certain embodiments, the modifying of the gut microbiota comprises treatment of the subject prior to treatment with the immune agonist antibody. In certain embodiments, the modifying of the gut microbiota comprises treatment of the subject concurrently with treatment with the immune agonist antibody. In certain embodiments, the modifying of the gut microbiota comprises treatment of the subject following treatment with the immune agonist antibody.

In certain embodiments, the modifying of the gut microbiota comprises treatment of the subject prior to and concurrently with treatment with the immune agonist antibody. In certain embodiments, the modifying of the gut microbiota comprises treatment of the subject prior to and following treatment with the immune agonist antibody. In certain embodiments, the modifying of the gut microbiota comprises treatment of the subject concurrently with and following treatment with the immune agonist antibody.

In certain embodiments, the method comprises assessing the susceptibility of the subject to the one or more side effects associated with the immune agonist antibody immunotherapy by assessing the abundance of one or more specific microorganisms in the gut microbiota of the subject. In certain embodiments, the method comprises assessing the susceptibility of the subject to the one or more side effects associated with the immune agonist antibody immunotherapy by assessing the abundance of one or more specific species or strains of bacteria in the gut microbiota of the subject.

Methods for assessing the abundance of one or more specific species or strains of bacteria in the gut microbiota of the subject are known in the art, and include for example 16S rRNA sequencing, metagenome sequencing, targeted-PCR, culturing or a MALDI-Biotyper.

Certain embodiments of the present disclosure provide a method of increasing the dose of an immune agonist antibody administered to a subject for immunotherapy by reducing one or more side effects associated with the administration of the immune agonist antibody.

In certain embodiments, the dose of an immune agonist antibody administered to a subject is limited by one or more side effects caused by the immune agonist antibody.

In certain embodiments, the present disclosure provides a method of increasing the dose of an immune agonist antibody able to be administered to a subject for immunotherapy by reducing one or more side effects associated with the administration of the immune agonist antibody, the method comprising modifying the gut microbiota in the subject and thereby increasing the dose of the immune agonist antibody able to be administered to the subject.

Side effects associated with the administration of an immune agonist antibody are as described herein.

Methods and agents for modifying the gut microbiota in the subject are as described herein.

The immune agonist antibody may be administered to the subject alone, in combination with an immune checkpoint inhibitor, and/or in combination with other therapies.

Certain embodiments of the present disclosure provide a method of reducing one or more side effects associated with immunotherapy using an immune agonist antibody in a subject suffering from a cancer without substantially affecting efficacy of the immunotherapy.

In certain embodiments, the present disclosure provides a method of reducing one or more side effects associated with immunotherapy using an immune agonist antibody in a subject suffering from a cancer without substantially affecting efficacy of the immunotherapy, the method comprising modifying the gut microbiota in the subject and thereby reducing the one or more side effects associated with the immunotherapy in the subject without substantially affecting the efficacy of the immunotherapy.

Side effects associated with the administration of an immune agonist antibody are as described herein.

Methods and agents for modifying the gut microbiota in the subject are as described herein.

The immune agonist antibody may be used alone, in combination with an immune checkpoint inhibitor, or with other therapies.

In certain embodiments, the present disclosure provides a method of reducing one or more side effects associated with immunotherapy using an immune agonist antibody in a subject suffering from a cancer with only minimally affecting efficacy of the immunotherapy, the method comprising modifying the gut microbiota in the subject and thereby reducing the one or more side effects associated with the immunotherapy in the subject without substantially affecting the efficacy of the immunotherapy.

Side effects associated with the administration of an immune agonist antibody are as described herein.

Methods and agents for modifying the gut microbiota in the subject are as described herein.

The immune agonist antibody may be used alone, in combination with an immune checkpoint inhibitor, or with other therapies.

Certain embodiments of the present disclosure provide use of bacteria capable of modulating the toxicity of an immune agonist antibody in the preparation of a medicament for treating toxicity in a subject associated with immunotherapy using an immune agonist antibody.

Examples of bacteria for use in a medicament are as described herein

Methods for producing bacteria for use in a medicament are known in the art. In certain embodiments, the bacteria are provided in a medicament comprising live bacteria. In certain embodiments, the bacterial are provide in a medicament comprising pasteurised bacteria.

Methods for producing medicaments containing bacteria are known in the art. Formulations for use in medicaments are known and described in, for example, Remington's Pharmaceutical Sciences, 17th ed., Mack Publishing Company, Easton, Pa., 1985, which is incorporated herein by reference in its entirety.

A suitable concentration of bacteria for delivery to the subject may be selected. For example, a liquid or powdered composition for use as a medicament having 10⁶-10⁹ bacteria in a suitable carrier may be used.

Administration routes for the medicament are as described herein.

In certain embodiments, the medicament is an oral medicament or a rectal medicament. Bacteria may, for example, be delivered in a food, or in a capsule, tablet or powder.

Certain embodiments provide use of an agent capable of modifying the gut microbiota in the preparation of a medicament for reducing one or more side effects associated with immunotherapy using an immune agonist antibody.

An effective amount of the agent may be selected. Agents are as described herein. In certain embodiments, the agent comprises one or more of an antibiotic, probiotic bacteria, a prebiotic, one or more suitable foods and/or supplements, and bacteriophage.

Methods for delivering agents in medicaments are known in the art.

Certain embodiments of the present disclosure provide a method of treating a subject suffering from a cancer with an immune agonist antibody by modifying the gut bacteria of the subject.

In certain embodiments, the present disclosure provides a method of treating a subject suffering from a cancer with an immune agonist antibody, the method comprising
treating the subject with the immune agonist antibody; and
modifying the gut microbiota of the subject prior to and/or concurrently with treatment with the immune agonist antibody.

Methods and agents for treating a subject suffering from a cancer with an immune agonist antibody are known in the art. Immune agonist antibodies are as described herein. In certain embodiments, the method comprises treating the subject with an immune agonist antibody alone, in combination with an immune checkpoint inhibitor, or with other therapies.

Methods for modifying the gut microbiota are as described herein.

In certain embodiments, the modifying of the gut microbiota comprises one or more of administration to the subject of an effective amount of an antibiotic, administration to the subject of an effective amount of probiotic bacteria, a faecal transplant, administration of an effective amount of a prebiotic, administration of an effective amount of one or more suitable foods and/or supplements, and administration to the subject of an effective amount of suitable bacteriophage, as described herein.

In certain embodiments, the method comprises assessing the abundance of one or more specific species or strains of bacteria in the gut microbiota of the subject. In certain embodiments, method comprises assessing the susceptibility of the subject to one or more side effects and/or toxicity associated with the immune agonist antibody immunotherapy by assessing the abundance of one or more specific species or strains of bacteria in the gut microbiota of the subject.

Methods for assessing the abundance of specific species or strains of bacteria are as described herein.

In certain embodiments, the method is used to reduce one or more side effects in the subject associated with the treatment with the immune agonist antibody. Side effects are as described herein.

Certain embodiments of the present disclosure provide a medicament for preventing and/or treating one or more side effects in a subject associated with treatment with an immune agonist antibody.

In certain embodiments, the present disclosure provides a medicament for preventing and/or treating one or more side effects in a subject associated with treatment with an immune agonist antibody, the medicament comprising an effective amount of one or more bacteria capable of reducing one or more side effects associated with treatment with an immune agonist antibody and/or an agent for modifying the gut microbiota.

In certain embodiments, the treating of the subject comprises treating the subject with an immune agonist antibody alone, in combination with an immune checkpoint inhibitor, or with other therapies.

Bacteria for used in a medicament are as described herein. In certain embodiments, the medicament comprises at least 10⁶ bacteria, at least 10⁷ bacteria, at least 10⁸ bacteria, at least 10⁹ bacteria or at least 10¹⁰ bacteria.

In certain embodiments, the medicament further comprises an antibiotic, a probiotic, a prebiotic, one or more suitable foods and/or supplements, and/or suitable bacteriophage, as described herein.

In certain embodiments, the medicament is an oral medicament or a rectal medicament. Medicaments are as described herein.

Certain embodiments of the present disclosure provide a method of treating a subject suffering from a cancer using an immune agonist antibody, the method comprising administering to the subject a medicament as described herein.

In certain embodiments, the medicament is used to treat a subject being treated with an immune agonist antibody alone, in combination with an immune checkpoint inhibitor, or with other therapies.

Certain embodiments provide a combination product for treating a subject with an immune agonist antibody.

In certain embodiments, the present disclosure provides a combination product for treating a subject with an immune agonist antibody, the product comprising the following components:
an immune agonist antibody; and
one or more bacteria capable of modifying the gut microbiota and/or an agent capable of modifying the gut microbiota.

Typically, the components contain an effective amount of the active agents to treat the subject. The components may be in a suitable form for administration directly to a subject, or in a form suitable for storage, and may be provided separately or on combination.

In certain embodiments, the immune agonist antibody comprises one or more of an anti-CD137 antibody, an anti-CD40 antibody, an anti-CD28 antibody, an anti-GITR antibody, an anti-OX40 antibody, an anti-CD27 antibody, and an anti-ICOS antibody.

The immune agonist antibody may be used alone, in combination with an immune checkpoint inhibitor, or with other therapies.

Bacteria capable of modifying the gut microbiota, and the dosages thereof, are as described herein.

Agents for modifying the gut microbiota are as described herein. In certain embodiments, the agent comprises an antibiotic, a prebiotic, one or more suitable foods and/or supplements, and/or suitable bacteriophage.

Certain embodiments of the present disclosure provide a method of assessing the susceptibility of a subject to one or more side effects associated with immunotherapy using an immune agonist antibody.

In certain embodiments, the present disclosure provides a method of assessing the susceptibility of a subject to one or more side effects associated with immunotherapy using an immune agonist antibody, the method comprising assessing the abundance of one or more specific species or strains of bacteria in the gut microbiota of the subject and thereby assessing the susceptibility of the subject to the one or more side effects associated with the immunotherapy using the immune agonist antibody.

Methods for assessing the abundance of a species or strain of bacteria in the gut microbiota are known in the art and also described herein.

The immunotherapy with the immune agonist antibody may involve immunotherapy with the immune agonist alone, in combination with an immune checkpoint inhibitor, or with other therapies.

In certain embodiments, a specific concentration of specific bacteria is indicative of an increased susceptibility or likelihood to one or more side effects. In certain embodiments, a specific concentration of specific bacteria is indicative of a decreased susceptibility or likelihood to one or more side effects.

In certain embodiments, a ratio of concentrations of one type of bacteria to another type of bacteria is indicative of an increased or decreased susceptibility or likelihood to one or more side effects.

Certain embodiments of the present disclosure provide a method of identifying a species or strain of bacteria capable of reducing one or more side effects associated with immunotherapy using an immune agonist antibody.

In certain embodiments, the present disclosure provides a method of identifying a species or strain of bacteria capable of reducing one or more side effects associated with immunotherapy using an immune agonist antibody, the method comprising:
altering the concentration of one or more types of candidate species or strains of bacteria in the gut of a subject; and
determining the ability of the change of the concentration of the candidate species or strain of bacteria to reduce one or more side effects associated with the immunotherapy;
thereby identifying the one or more types of candidate species or strains of bacteria as a species of bacteria capable of reducing one or more side effects associated with immunotherapy using an immune agonist antibody.

In certain embodiments, the method comprises the use of an animal model to identify the species or strains of bacteria capable of reducing one or more side effects associated with immunotherapy.

In certain embodiments, the method comprises administering one or more species or strains of bacteria to identify the bacteria as a species capable of reducing the one or side effects.

In certain embodiments, the method comprises depletion of one or more species or strains of bacteria to identify the bacteria.

In certain embodiments, the method comprises use of a clinical trial to identify the species or strains of bacteria capable of reducing one or more side effects associated with immunotherapy.

Certain embodiments of the present disclosure provide a method of identifying an agent capable of reducing one or more side effects associated with immunotherapy using an immune agonist antibody.

In certain embodiments, the present disclosure provides a method of identifying an agent capable of reducing one or more side effects associated with immunotherapy using an immune agonist antibody, the method comprising:
administering a candidate agent capable of modifying the gut microbiota to a subject; and
determining the ability of the candidate agent to reduce a side effect associated with the immunotherapy,
thereby identifying the candidate agent as an agent capable of reducing a side effect associated with immunotherapy using an immune agonist.

In certain embodiments the method comprises use of an animal model to identify the candidate agent.

Examples of candidate agents include bacteria, a drug, a small molecule, a protein, a virus, a bacteriophage, a polypeptide, a nucleic acid, a lipid, a ligand, a lipid, a carbohydrate, a nucleic acid, an oligonucleotide, a ribozyme, a biologic, an aptamer, a peptide, a cofactor, a ligand, a ligand mimetic, a receptor, an enzyme, a metal ion, a chelate, a nucleic acid, and an antibody or an antigen binding part thereof. Other types of agents are contemplated.

In certain embodiments, the method comprises use of a clinical trial to identify the agent capable of reducing one or more side effects associated with immunotherapy.

Standard techniques may be used for microbiology, cell culture, molecular biology, recombinant DNA technology, tissue culture and transfection. The foregoing techniques and other procedures may be generally performed according to conventional methods well known in the art and as described in various general and more specific references that are cited and discussed throughout the present specification. See e.g., Sambrook et al. Molecular Cloning: A Laboratory Manual (4th ed., Cold Spring Harbor Laboratory Press, Cold Spring Harbor, N.Y. (2012) and Ausubel et al Current Protocols in Molecular Biology (2012) John Wiley & Sons, both of which are herein incorporated by reference.

The present disclosure is further described by the following examples. It is to be understood that the following description is for the purpose of describing particular embodiments only and is not intended to be limiting with respect to the above description.

### Example 1—the Gut Microbiota Modulates the Toxicity of Immune Agonist Antibody Cancer Immunotherapies

Summary

Immune agonist antibodies (IAAs) are promising cancer immunotherapies that target immune co-stimulatory receptors, such as CD40 and CD137, on a broad range of immune cells, inducing tumor immune cell infiltration, activating direct anti-tumor immunity, and increasing sensitivity to immune checkpoint inhibitors (ICI). A major obstacle to their clinical translation, however, is dose-limiting toxicity and in a substantial proportion of patients, serious immune-mediated side effects including high grade and sometimes fatal liver damage, cytokine release and colitis. We found that the gut microbiota plays a critical role in mediating the immunotoxicity induced by the IAAs, anti-CD40 and anti-CD137. Germ-free or antibiotic-treated mice had significantly reduced IAA-induced cytokine release and were protected from organ damage induced by these therapies. Importantly, there was minimal impact on IAA anti-tumor efficacy, alone or in combination with the ICI, anti-PD1. Our results suggest microbiota-targeted therapies as a new approach to overcome the toxicity associated with IAAs.

Introduction

The development of immune checkpoint inhibitors (ICIs) against PD1/PDL1 and CTLA4 have revolutionized the treatment of cancer. ICIs can induce long-term, potentially curative, responses in some patients, particularly those suffering from advanced melanoma. Unfortunately, these outstanding results are only observed in a relatively small portion of patients and their activity is limited mostly to tumors types with a strong immune infiltrate. A recent study found that while 40% of all oncology patients in the USA are eligible for ICI therapy, less than 13% will respond to treatment. To improve response rates, approaches which combine ICIs with other immunotherapies, such as immune agonist antibodies (IAAs), are currently being evaluated in ongoing clinical trials. Unlike ICIs which block T cell suppressive pathways, IAAs target immune co-activating/co-stimulatory receptors such as CD40, CD137, CD28, CD27, GITR or OX40, which are expressed on a range of different myeloid and lymphoid cell populations. IAA binding to these receptors provides activating signals that induce a range of downstream cellular processes including enhanced immune cell proliferation, survival, antigen presentation and cytotoxicity. IAAs are therefore attractive cancer immunotherapies as they induce both direct anti-tumor immune responses and increase immune cell infiltration into tumors, boosting the efficacy of ICIs. Correspondingly, there are 70 recruiting or upcoming phase I-III clinical trials evaluating IAAs, predominantly in combination with other therapies such as ICIs (clinicaltrials.gov).

As the co-activating receptors targeted by IAAs are critical modulators of immune function, IAAs also activate the immune system more broadly, leading to a range of undesirable immune-related adverse events in patients. Clinical trials of IAAs have been hampered by immunotoxicity, ranging from dose-limiting toxicities, to serious (grade 3 or higher) immune-mediated side effects in some patients (˜15-30%) including cytokine release syndrome (CRS), liver damage, and even death. For example, clinical trials of several different CD40 agonists (ChiLob7/4, CP-870,893) have identified dose-limiting toxicity due to CRS and grade 3-4 hematological and liver toxicities. Trials of another IAA targeting CD137 (urelumab), were halted due to severe dose-dependent liver toxicity, while anti-CD28 was used in a catastrophic Phase I trial where every patient required long-term hospitalization due to severe CRS. Several strategies are now being trialed to manage the toxicity of IAAs including limiting the dose, co-administering immunosuppressive corticosteroids, or administering immune agonists locally to tumors. However, many of these strategies could come at the expense of anti-tumor efficacy by non-specifically dampening immune responses to therapy.

As the liver is a common site of IAA toxicity, we hypothesized that the gut microbiota could play a critical role in mediating immunotoxicity induced by IAAs, particularly in the liver. Here, we report that antibiotic treated or germ-free mice were protected against the liver damage and cytokine release syndrome induced by treatment with the IAAs, anti-CD40 or anti-CD137. Importantly, antibiotic treatment had only a minor effect on the anti-tumor efficacy of IAAs, alone or in combination with the ICI, anti-PD1. Our data provides strong rationale to investigate if the composition of the microbiota can predict patients at risk of high grade immune agonist induced immunotoxicity and suggest that microbiota-targeted interventions could substantially reduce the immunotoxicity associated with IAAs, overcoming a critical roadblock to their clinical application.

Results

Antibiotic Treatment Reduces the Immunotoxcity of Anti-CD40 without Reducing its Anti-Tumor Efficacy.

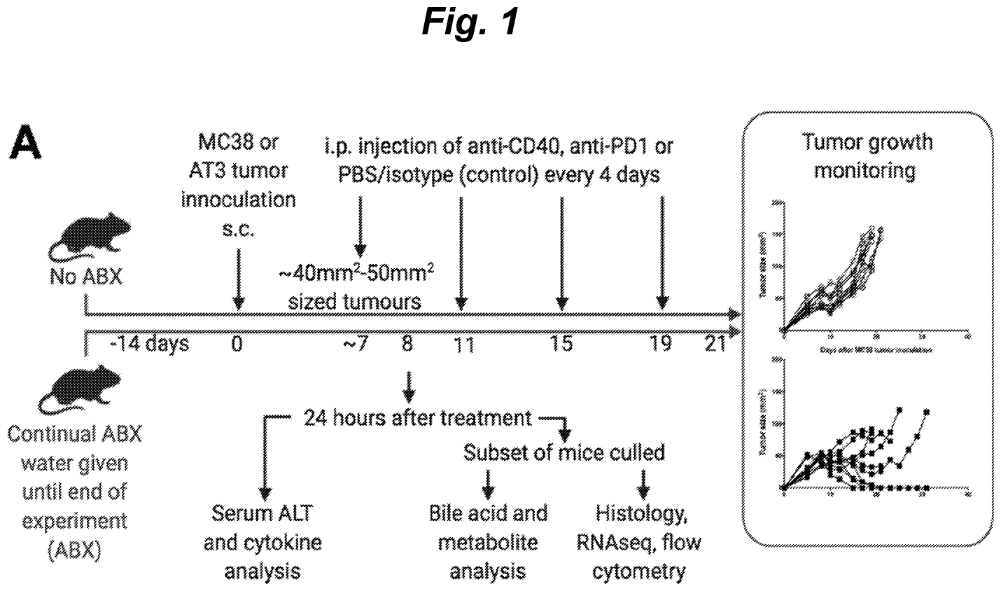

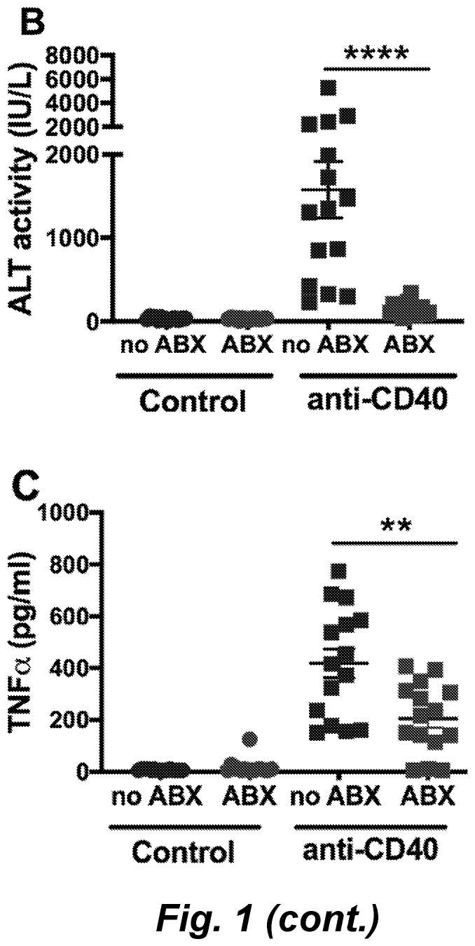

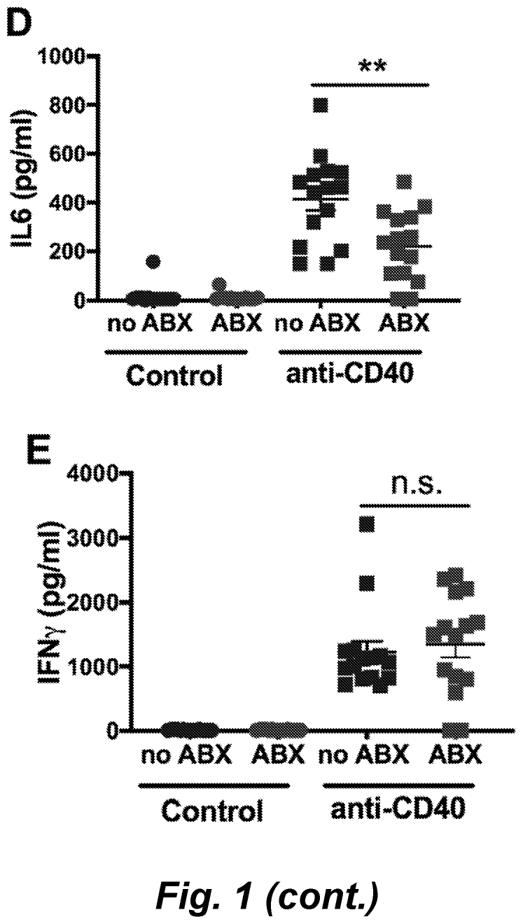

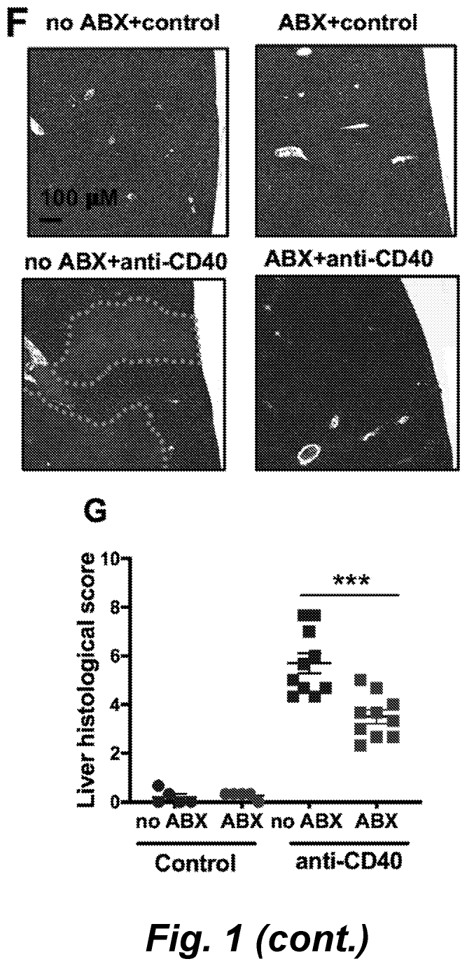

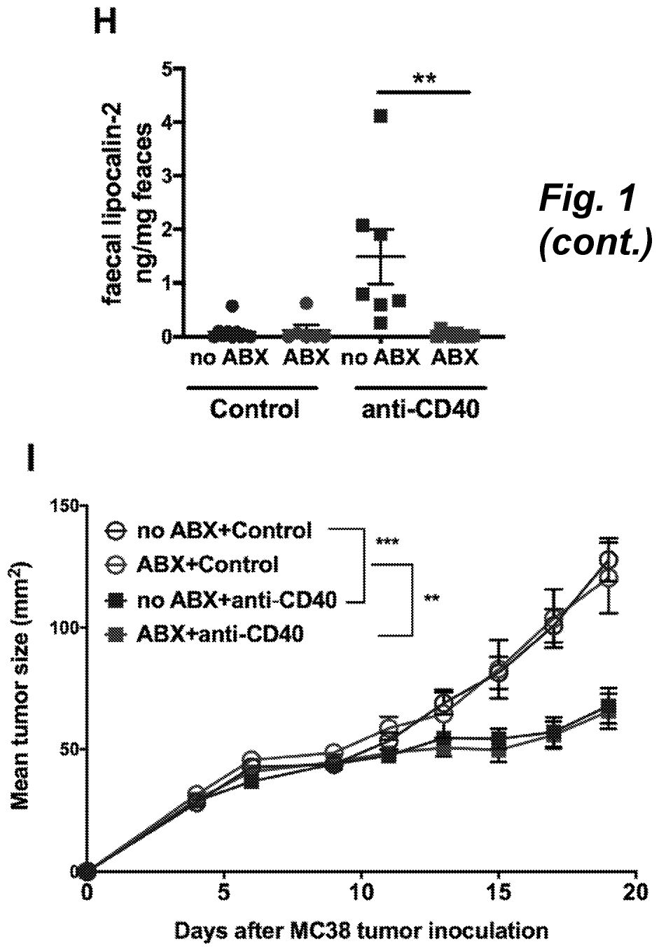

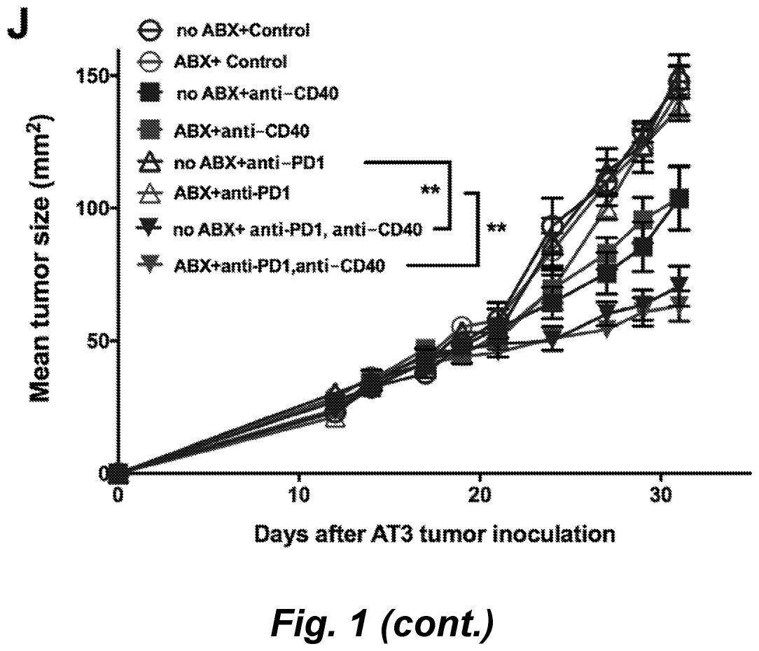

Immune-mediated liver toxicity indicated by elevated liver enzymes in serum is one of the most common severe (≥grade 3) side effects observed with anti-CD40 treatment. Approximately 75% of the liver's blood supply is provided by the hepatic portal vein, which first passes through the gastrointestinal tract and carries a constant supply of bacterial products from the gut microbiota. We hypothesized that the liver toxicity and systemic cytokine release syndrome (CRS) associated with anti-CD40 could be modulated by signals from the gut microbiota. To test this hypothesis, we investigated the impact of antibiotic treatment (ABX) on responses to the IAA, anti-CD40, in tumor-bearing C57BL/6 mice (FIG. 1A). Mice were treated via their drinking water with ampicillin and neomycin, starting ˜2 weeks prior to tumor inoculation and continuing for the duration of the experiment. These antibiotics have a broad spectrum of activity and reduced fecal bacterial load by ˜4 log-fold. In normally colonized specific and opportunistic pathogen free (SOPF) mice, treatment with anti-CD40 induced a rapid onset cytokine storm and liver damage that peaked 24 hours post therapy initiation. Serum alanine aminotransferase (ALT), a marker of liver damage was significantly elevated following anti-CD40 treatment and was almost completely inhibited in ABX-treated mice (FIG. 1B). ABX-treated mice also had significantly reduced levels of the pro-inflammatory cytokines, IL6 and TNFα, in serum following anti-CD40 treatment (FIG. 1C-D). Anti-CD40 treatment also induced elevated IFNγ levels in serum, a key lymphocyte effector cytokine that is important for anti-tumor responses, which was not reduced by ABX treatment (FIG. 1E). Histological analysis of livers collected 24 hours after anti-CD40 treatment revealed large areas, some wedge-shaped, of hepatocellular necrosis of varying size, with no discernible zonal pattern. A few necrotic areas were associated with thrombi in portal blood vessels, particularly portal veins, and all were dramatically reduced by ABX treatment, confirmed when livers were scored against published standards (FIG. 1F-G).

We also assessed whether antibiotic treatment reduced anti-CD40-induced tissue damage in other organs. We observed that anti-CD40 treated mice were reluctant to produce stool during monitoring 24 hours after treatment, suggesting that anti-CD40 could induce gastro-intestinal inflammation as has been reported clinically. Histological analysis of no ABX+ anti-CD40 treated colons identified numerous enterocyte-derived apoptotic bodies, particularly in the germinative (stem cell) region in the basal area of the glands and an observed increase in lymphocytes in the lamina propria, with intraepithelial T-cells were markedly increased in areas of substantial colonic injury. There was sometimes abundant mucus extrusion from goblet cells into the lumen and, in some segments, enterocytes were hyperplastic. While similar lesions were observed in ABX+anti-CD40 treated colons, they were much less severe and reduced in distribution within the colon. To quantitate damage to the colon, we assessed fecal lipocalin-2 levels in stool samples as a marker of intestinal inflammation Anti-CD40 treated mice had significantly elevated levels of lipocalin-2 in their stool samples and this was almost completely inhibited by ABX treatment (FIG. 1H). Histological analysis of lung or skin sections indicated that anti-CD40 did not significantly increase immune infiltration or damage in these organs.

To investigate whether ABX treatment altered the anti-tumor efficacy of anti-CD40 against established transplanted subcutaneous tumors, we inoculated mice treated with or without antibiotics with the MC38 colorectal cancer cell line or the AT-3 triple negative breast cancer cell line. Tumors were allowed to establish until ˜40-50 mm² in size, before treatment with anti-CD40 and/or anti-PD1 was initiated. Critically, we found that ABX treatment had no impact on the efficacy of anti-CD40 in suppressing MC38 tumor growth (FIG. 1). Due to their ability to prime potent anti-tumor immune responses, IAAs are being actively investigated in combination with other anti-cancer therapies. In particular, there is significant pre-clinical evidence that IAAs strongly synergize with ICIs, which was also reported in results from early phase I clinical trials. As the efficacy of ICIs, such as anti-PD1, has been shown to be strongly reduced by ABX exposure, we evaluated the impact of ABX on the efficacy of anti-CD40/anti-PD1 combination therapy against the anti-PD1-insensitive AT-3 tumors. As expected, anti-PD1 was completely ineffective at suppressing AT-3 tumor growth alone. Treating mice with a single dose of anti-CD40 was sufficient to sensitize AT-3 tumors to anti-PD1 and tumor growth was potently suppressed by the anti-CD40/anti-PD1 combination therapy. Furthermore, the efficacy of anti-CD40/anti-PD1 combination therapy was not altered by ABX treatment (FIG. 1J).

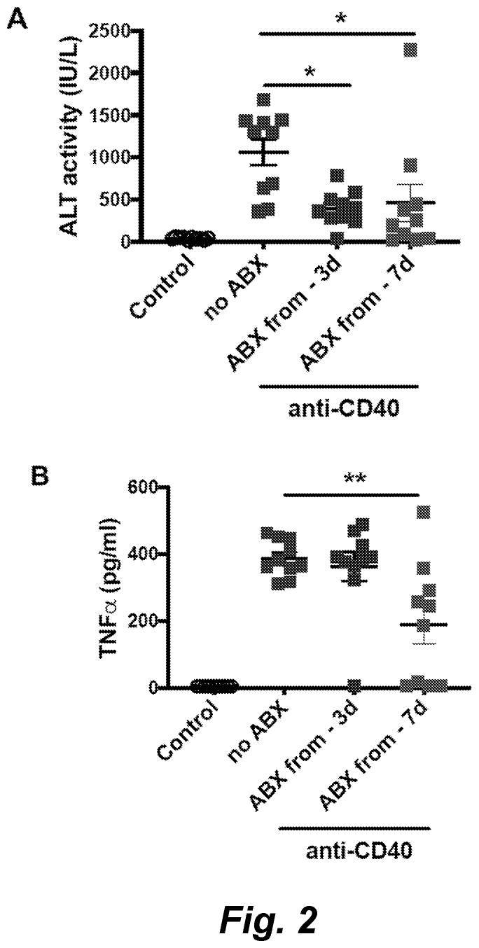

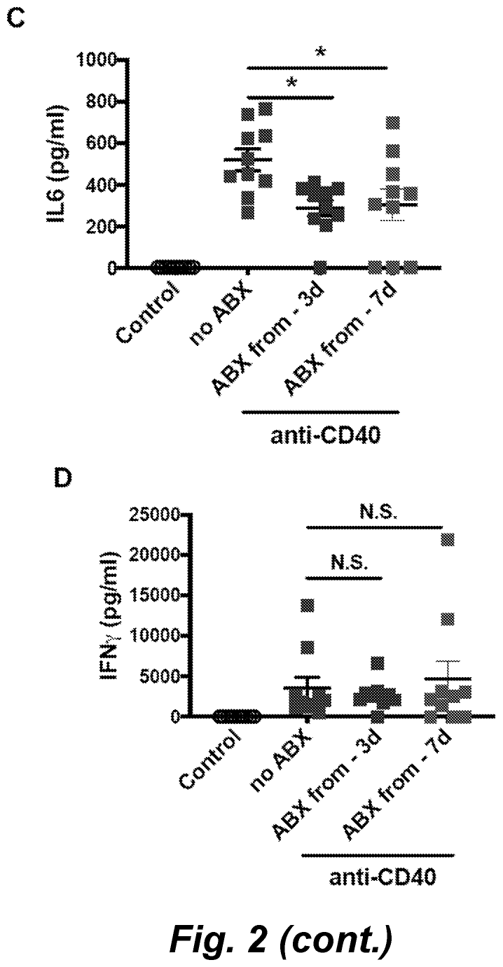

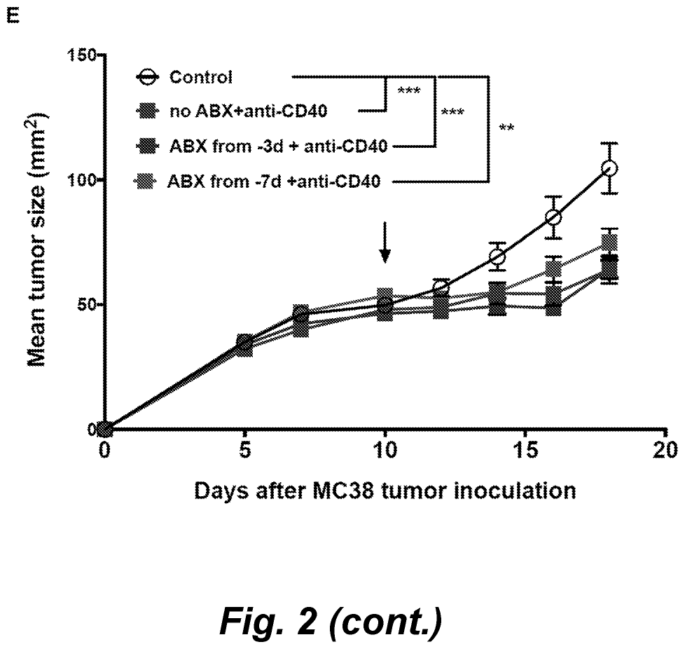

In the experiments described above, mice were administered ABX prior to tumor inoculation, meaning that they were exposed to ABX for ˜3 weeks before anti-CD40 treatment. We next investigated whether a shorter course of ABX, starting only 3 or 7 days before the first anti-CD40 treatment was sufficient reduce immunotoxicity. Again, we observed that mice treated with ABX starting 3 or 7 days before anti-CD40 treatment had significantly reduced ALT levels (FIG. 2A). Serum TNFα was only significantly reduced following 7 days ABX treatment (FIG. 2B). Anti-CD40-induced serum IL6 was significantly reduced in mice treated with ABX 3 or 7 days before anti-CD40 treatment (FIG. 2C). As we previously observed, IFNγ levels in serum were not altered by antibiotic treatment (FIG. 2D). ABX starting 3 or 7 days before anti-CD40 treatment also had no impact on the anti-tumor efficacy of anti-CD40 against MC38 tumors (FIG. 2E). Overall, these data indicate that antibiotic treatment potently reduces the immunotoxicity of anti-CD40, without impacting its anti-tumor efficacy against MC38 or AT-3 subcutaneous tumors, alone or combined with anti-PD1.

Antibiotic Treatment Also Reduces the Immunotoxicity Associated with Anti-CD137 Treatment

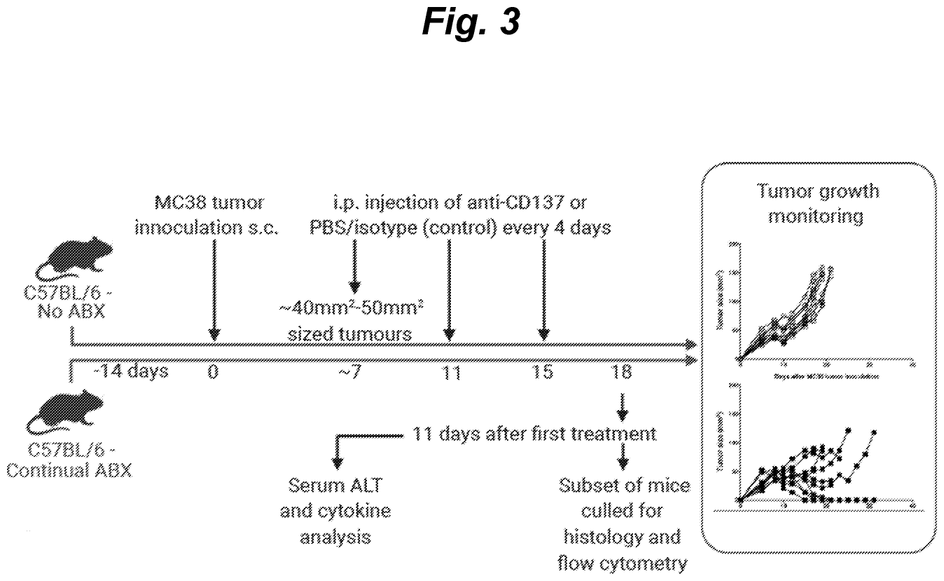

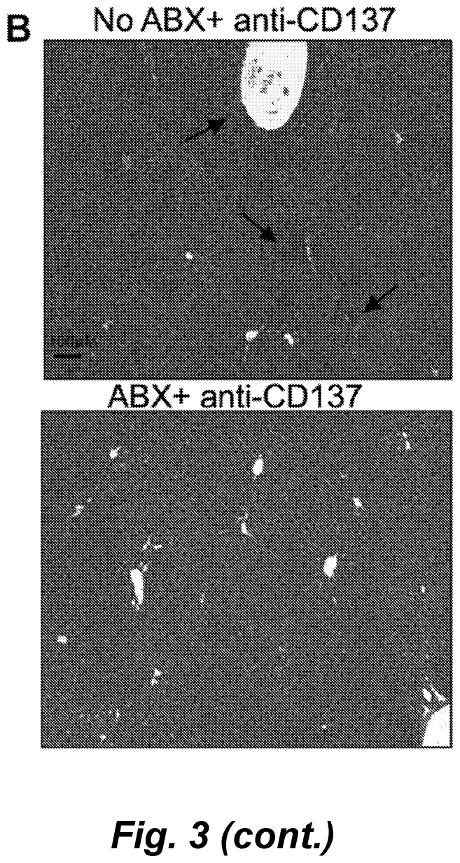

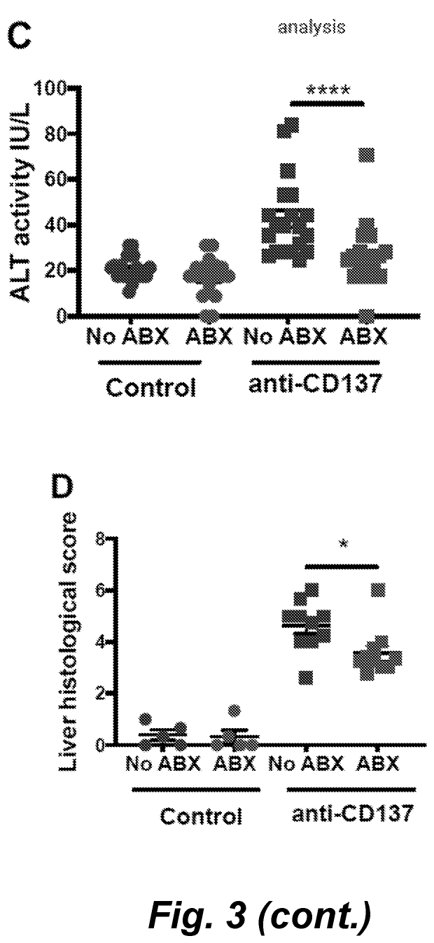

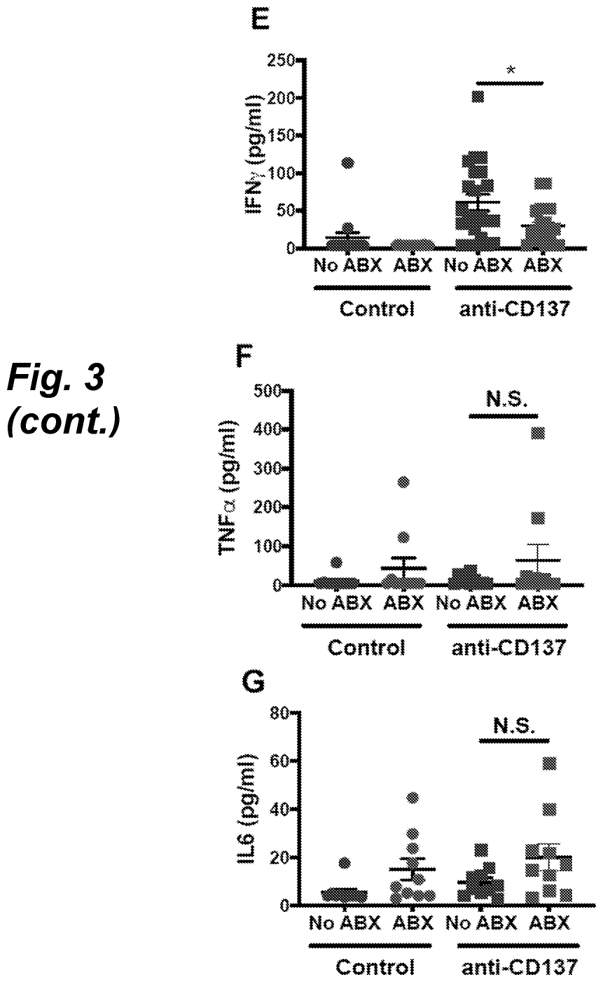

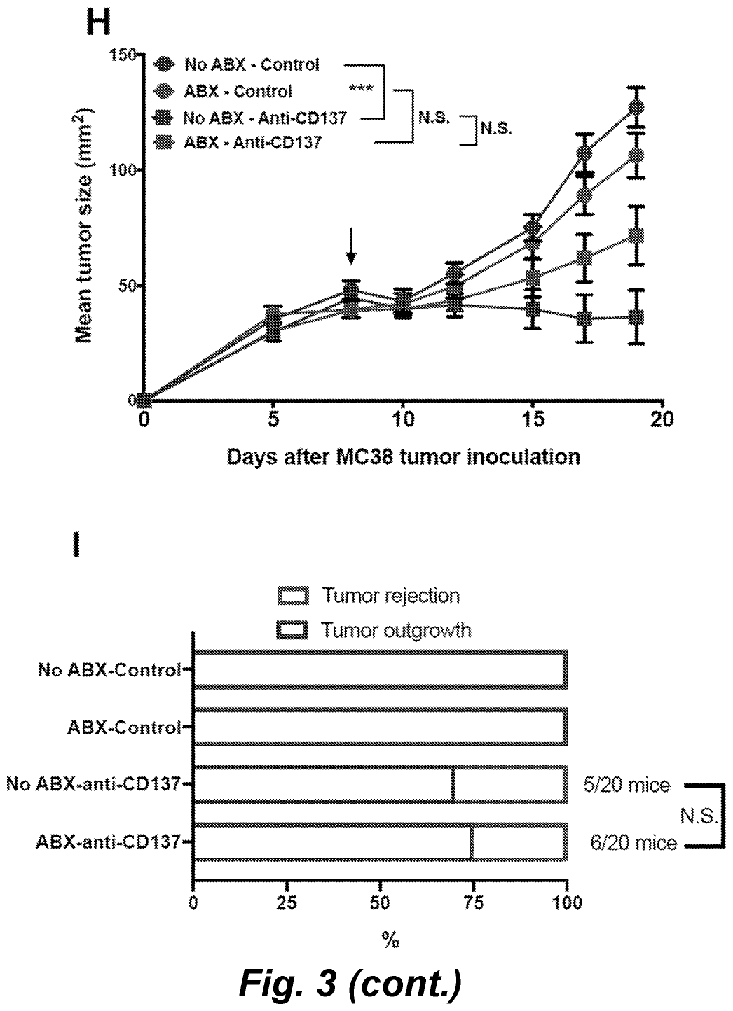

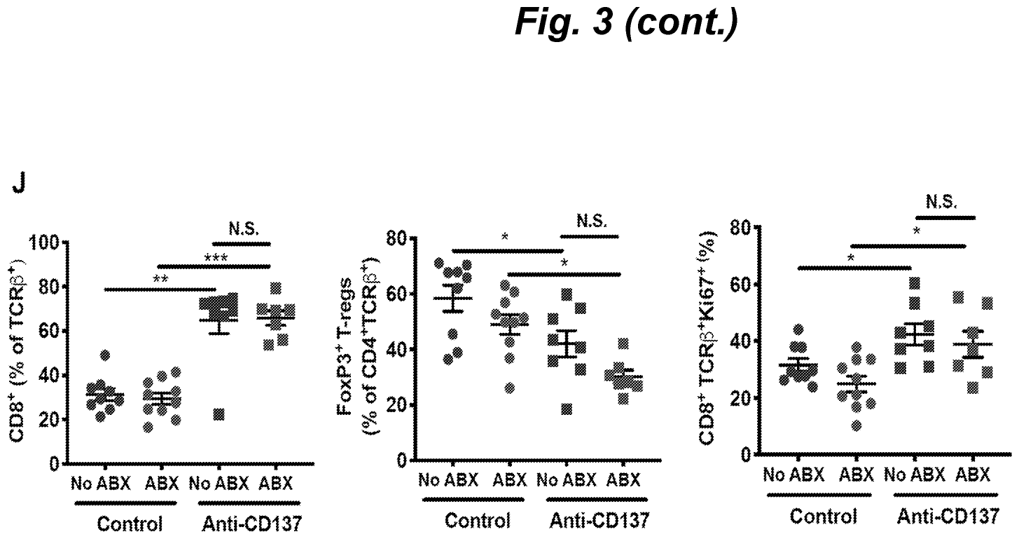

Urelumab, an anti-CD137 IAA, induces severe (grade 3 and above) liver damage in patients, particularly when used at higher doses. We therefore investigated whether antibiotic treatment also altered the immunotoxicity associated with anti-CD137 treatment (FIG. 3A). Consistent with previous reports, anti-CD137 treatment induced liver damage peaking at 11 days post treatment initiation, as indicated by significantly elevated serum ALT levels and immune cell infiltration into the liver (FIG. 3B-C). Consistent with what we observed for anti-CD40, anti-CD137-induced serum ALT levels were significantly reduced in ABX mice (FIG. 3C) and histological analysis of the livers showed that antibiotic treatment significantly reduced liver damage induced by anti-CD137 (FIG. 3D). While CRS is not a typical side effect of anti-CD137 treatment, we found that serum IFNγ, which was induced by anti-CD137 treatment, was significantly reduced by ABX treatment (FIG. 3E). TNFα or IL6 levels in serum were not significantly elevated in mice in response to anti-CD137 treatment (FIG. 3F-G). In contrast to anti-CD40, antibiotic treatment was observed to reduce the anti-tumor efficacy of anti-CD137 against MC38 tumors (FIG. 3H), although the rate of tumor rejection was not significantly altered (FIG. 3I). Flow cytometry analysis of immune cell infiltration into tumors revealed that antibiotic treatment did not significantly alter tumor immune cell infiltration or activation of cytotoxic CD8+ T cells after anti-CD137 treatment (FIG. 3J). Interestingly, regulatory T cells were lower in ABX mice treated with anti-CD137, though these data did not reach statistical significance (FIG. 3J). Taken together, these data indicate that antibiotic treatment potently reduces the immunotoxicity of anti-CD137, while also having a minor impact on its anti-tumor efficacy.

Germ-Free Mice have Significantly Reduced Anti-CD40 and Anti-CD137 Induced Immunotoxicity which is Restored by Fecal Microbiota Transplant

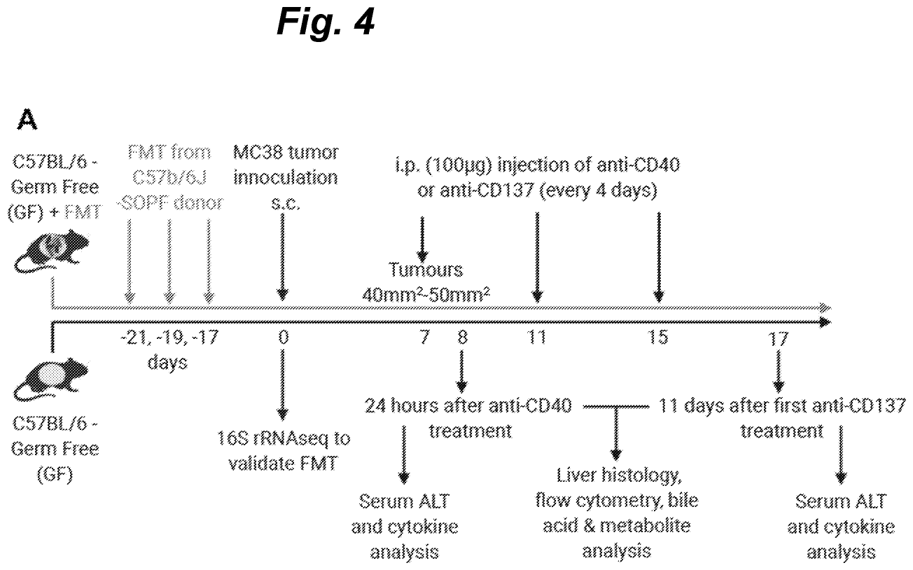

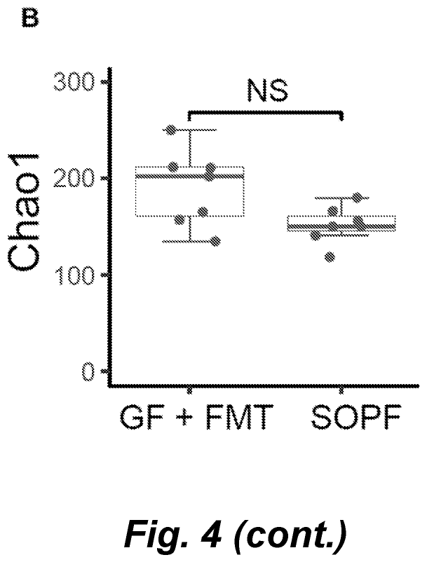

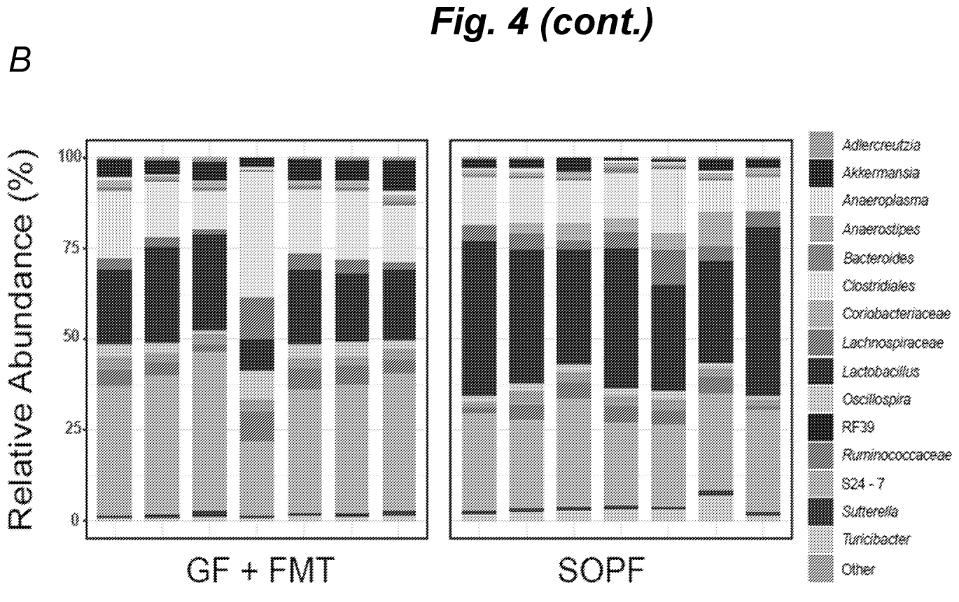

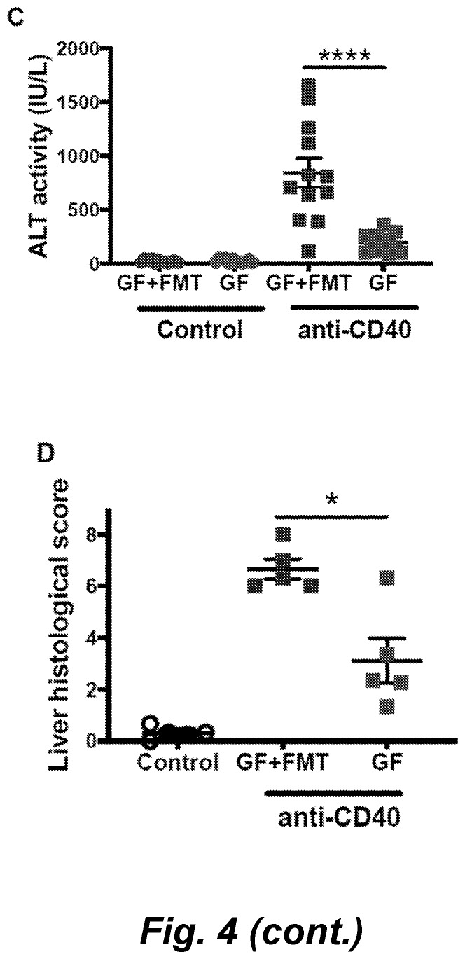

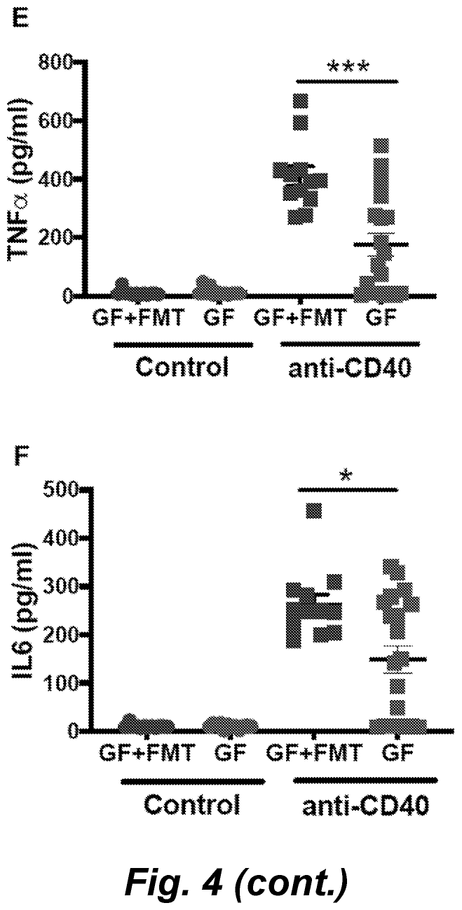

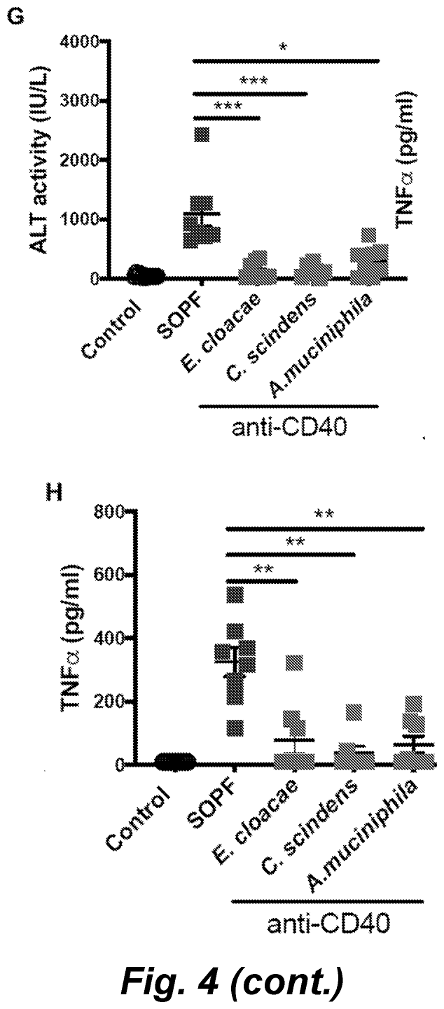

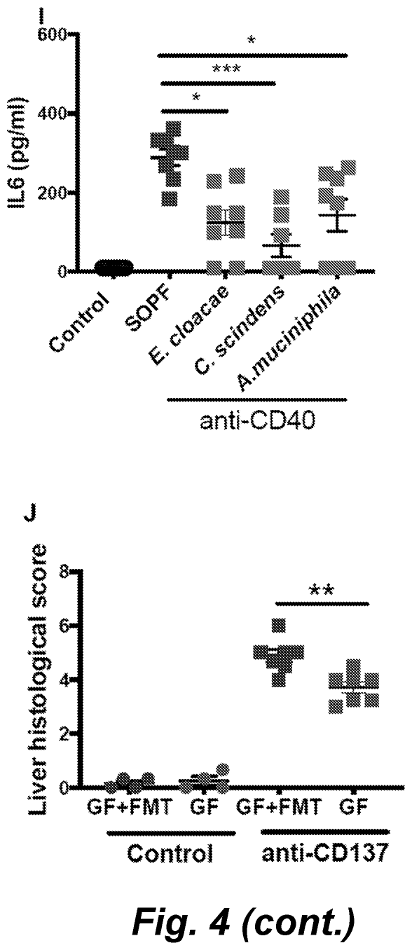

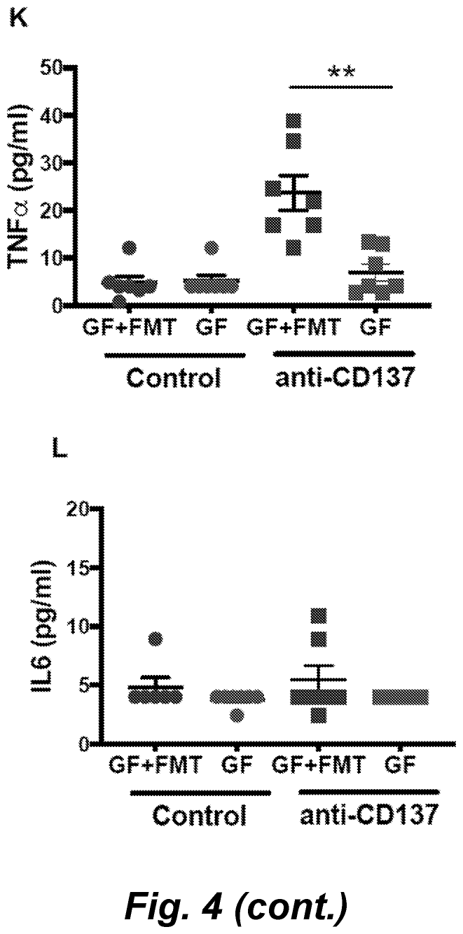

To demonstrate that the effects of antibiotic treatment on anti-CD40 and anti-CD137 induced immunotoxicity was mediated via the gut microbiota, we investigated responses to anti-CD40 and anti-CD137 in germ-free (GF) mice and in GF mice recolonized by fecal microbiota transplant (GF+FMT) from SOPF mice. Successful colonization of GF mice by FMT was confirmed by 16S rRNA gene sequencing of fecal samples collected 3 weeks after the first FMT, which demonstrated that the microbiota of GF+FMT mice had a similar composition and diversity to that of SOPF mice (FIG. 4B). Similar to ABX-treated mice, GF mice had significantly reduced liver damage (serum ALT and histological score) following anti-CD40 treatment, while FMT from donor SOPF mice restored anti-CD40-induced liver damage in GF mice (FIG. 4C-D). GF mice also had significantly reduced serum TNFα (FIG. 4E) and IL6 (FIG. 4F) levels compared to GF+FMT mice. As we had observed in ABX-treated SOPF mice, anti-CD40 induced IFNγ levels in serum were not significantly different between GF and GF+FMT mice (data not shown). Anti-CD137 induced liver damage and the levels of TNFα and IFNγ in serum were also significantly reduced in GF mice compared to GF mice re-colonized with an FMT from SOPF mice (FIG. 4J-K, data not shown). Serum IL6 was not significantly increased in any of the anti-CD137 treated groups (FIG. 4L). Taken together, these data demonstrate that the gut microbiota plays a causative role in mediating the immunotoxicity induced by the IAAs, anti-CD40 and anti-CD137.

To investigate whether specific taxa in the microbiota are sufficient to enhance anti-CD40 induced immunotoxicity, we mono-colonized GF mice with Enterobacter cloacae, a Gram negative LPS-producing commensal, Clostridium scindens, which is known to synthesize secondary bile acids from secreted primary bile acids, or Akkermansia muciniphila, a bacterium shown to improve activity of anti-PD1/PDL1 ICIs. Mice were gavaged twice with ˜1×10⁷ of each monoculture and colonization confirmed by 16S rRNA SANGER sequencing. Once colonization was confirmed, mice were treated with anti-CD40 and 24 hours later, serum ALT, and cytokines assessed. None of these taxa restored anti-CD40 induced serum ALT to levels observed in anti-CD40 treated SOPF mice (FIG. 4G). However, serum TNFα and IL6 levels were also significantly lower in anti-CD40 treated mice colonized with these taxa, compared to anti-CD40 treated SOPF mice (FIG. 4H-1).

The Gut Microbiota Modulates Immune Cell Infiltration into the Liver Following Anti-CD40 or Anti-CD137 Treatment.

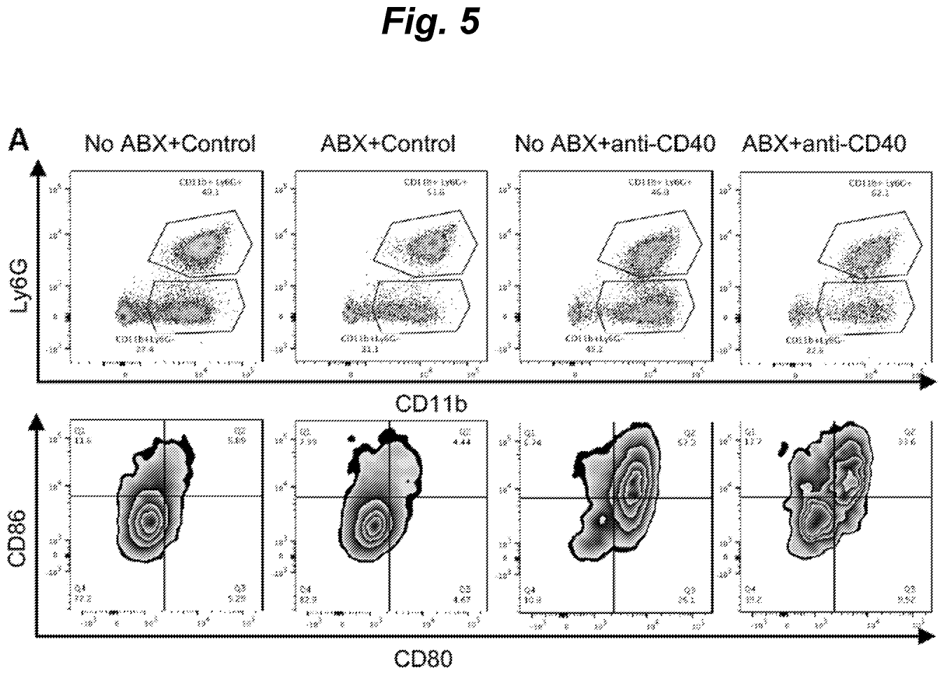

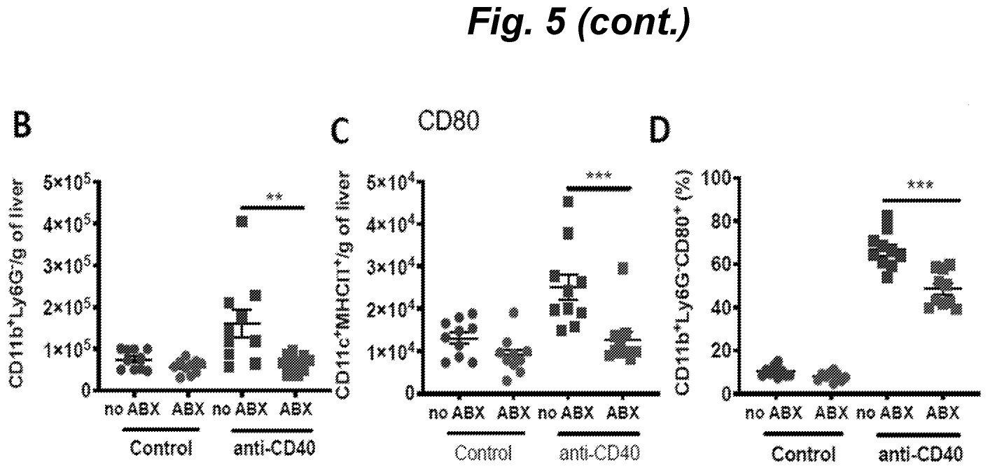

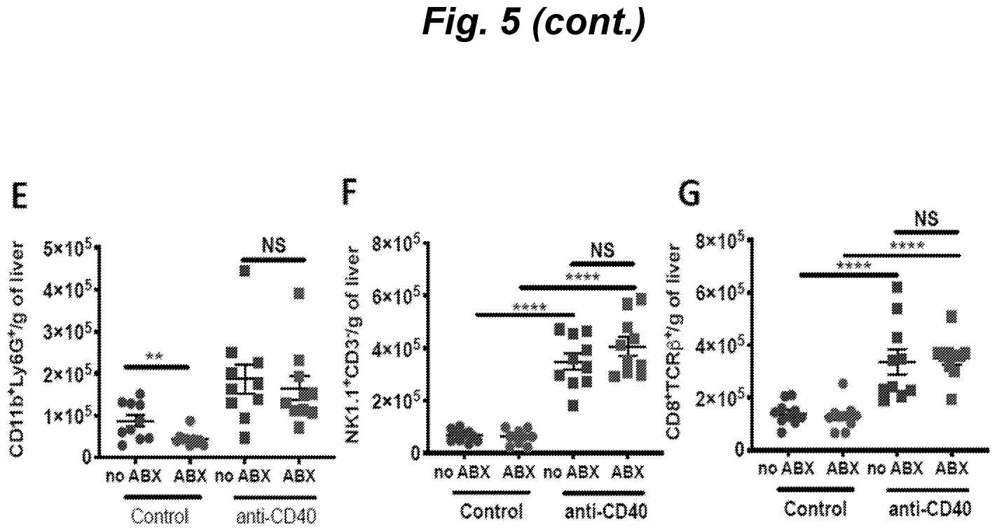

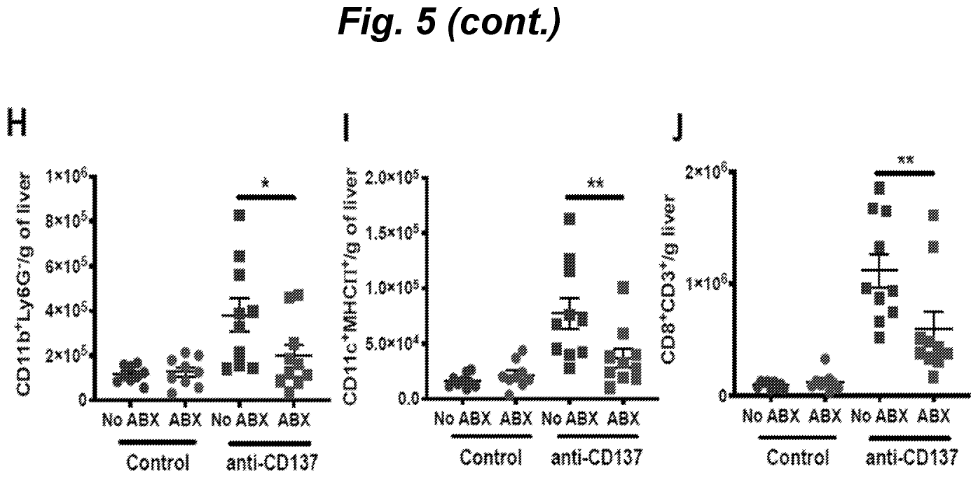

The liver damage induced by anti-CD40 and anti-CD137 has been previously shown to be driven by pathogenic immune responses in the liver. Anti-CD40 liver toxicity is driven by myeloid cells such as monocytes, macrophages, dendritic cells and neutrophils, while CD8⁺T cells are critical for anti-CD137 induced liver damage. These data suggested that the gut microbiota could modulate the liver damage induced by IAA treatment by altering immune cell infiltration and/or activation. To investigate this, we used flow cytometry to assess immune cell populations in livers collected 24 hours after the first anti-CD40 treatment (i.e. when anti-CD40-induced serum ALT was at its peak). Anti-CD40 treatment induced a rapid influx of immune cells into the liver, increasing the total CD45.2⁻ population in the liver by ˜50%. ABX treatment did not significantly alter the anti-CD40-induced infiltration of CD45.2⁺ cells into the liver, however, ABX treatment almost completely blocked the infiltration of CD11b⁺Ly6G⁻ (macrophages and monocytes) into the liver following anti-CD40 treatment (FIG. 5A-B). Anti-CD40 induced infiltration of conventional dendritic cells (CD11c⁺MHCII⁺ cDCs) was also strongly abrogated in ABX-treated mice (FIG. 5C). Furthermore, macrophages/monocytes and cDCs were less activated ABX-treated mice following anti-CD40 treatment, as indicated by significantly reduced CD80 and CD86 expression (FIG. 5A,D). Neutrophils (Cd11b⁺Ly6G⁺), NK-cells (NK1.1⁺CD3⁻) and CD8⁺ T-cells (CD8⁺TCRβ⁺) were significantly increased in livers of mice following anti-CD40 treatment, however, their infiltration was not significantly reduced by ABX treatment (FIG. 5E-G). NKT-like cells (NK1.1⁺CD3⁺) were significantly reduced following anti-CD40 treatment but were not significantly different between ABX and untreated mice (data not shown). CD4⁺ T cells (CD4⁺TCRβ⁺) were not significantly altered by anti-CD40 treatment (data not shown).

We also assessed immune cell infiltration in untreated and ABX-treated mice following anti-CD137 treatment Livers were collected for flow cytometry 11 days after anti-CD137 therapy commenced, when anti-CD137 induced ALT levels were at their peak. Similar to anti-CD40, ABX treatment significantly reduced anti-CD137 induced immune cell infiltration into the liver, most prominently monocytes/macrophages (FIG. 5H), cDCs (FIG. 5I) and CD8⁻ T cells (FIG. 5J). CD4⁺ T cells were also significantly reduced in ABX treated mice following anti-CD137 treatment, while NK-cells were potently depleted by anti-CD137 in both ABX-treated and untreated mice (data not shown). These data are consistent with reports that CD137 ligation causes activation of induced cell death in NK cells. Taken together, these data indicate that antibiotic treatment reduces the liver immunotoxicity induced by anti-CD40 and anti-CD137 by blocking signals from the microbiota that are required for pathogenic immune cell infiltration into the liver following IAA treatment.

Antibiotic Treatment Significantly Alters Anti-CD40 Induced Changes in Liver Gene Expression

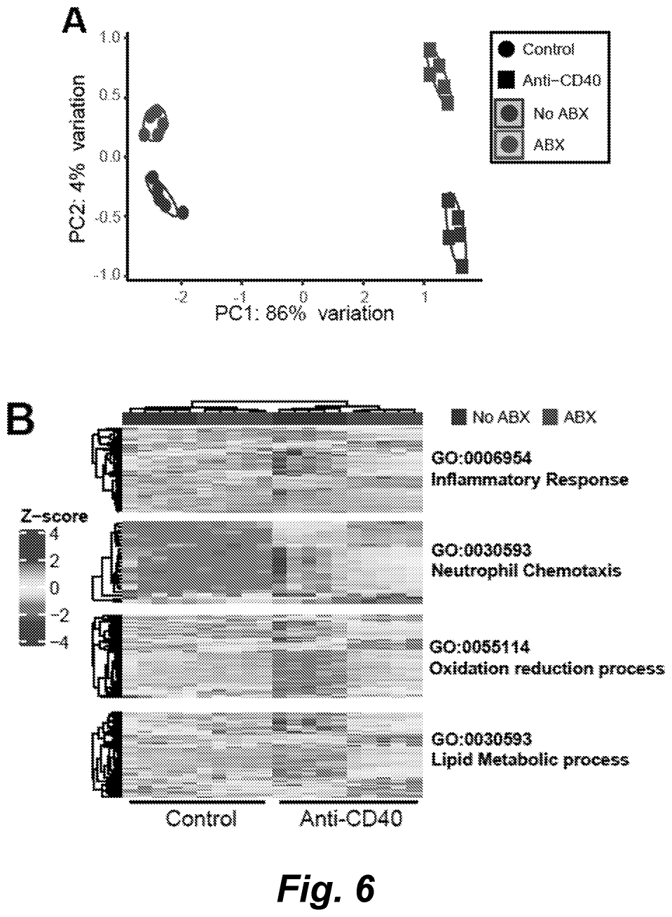

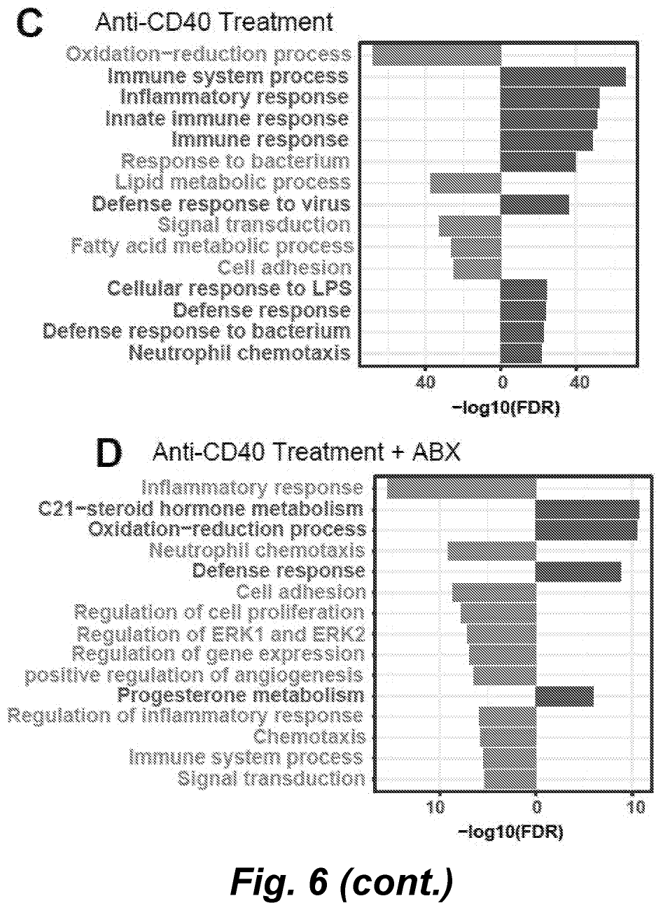

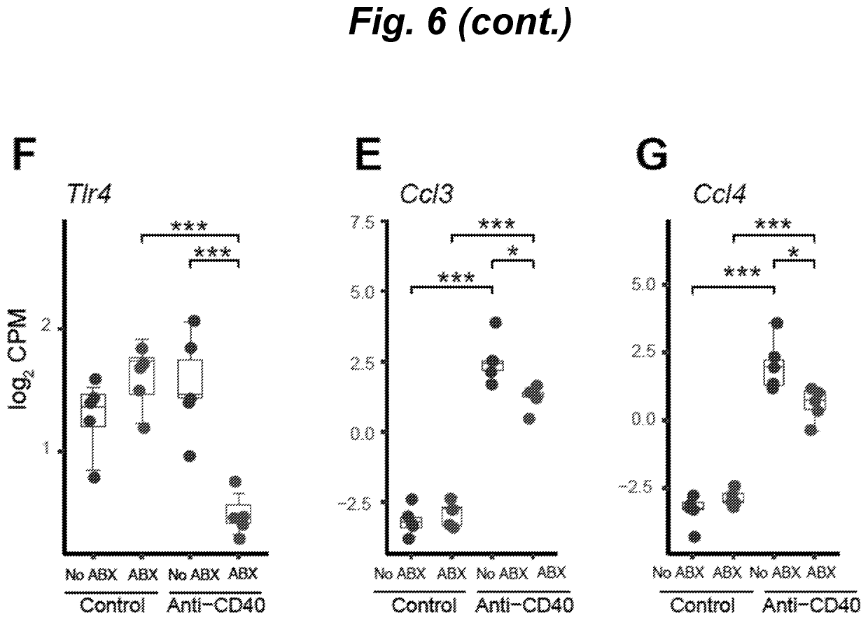

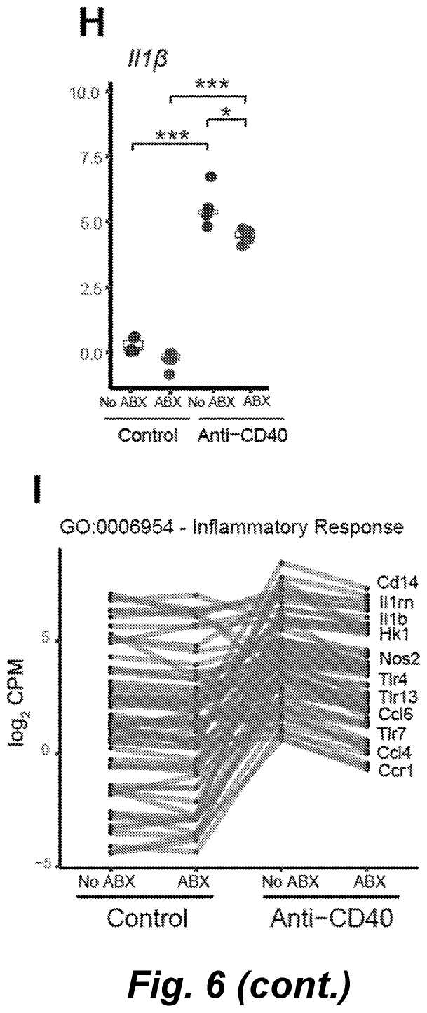

To further assess how antibiotic treatment modulates responses to anti-CD40 in the liver, gene expression in the livers of untreated and anti-CD40 treated mice (with and without antibiotic treatment) was profiled using RNA-Seq. A total of 1.18 billion reads were sequenced (mean 59 million single-end, 100-bp reads per sample). Multidimensional scaling analysis of the gene expression data revealed that the primary sources of variation in the data were driven by anti-CD40 treatment, followed by antibiotic treatment (FIG. 6A). Treatment with anti-CD40 profoundly altered gene expression in the liver, significantly changing the expression of more than 1,500 genes at least 2-fold (FDR<0.05), in comparison to PBS treated control mice. Pathway analysis revealed that anti-CD40 treatment induced dramatic changes in the expression of a broad array of immune and metabolic pathways (FIG. 6B-C) More specifically, anti-CD40 treatment led to a significant up-regulation of genes involved the immune and inflammatory responses including neutrophil degranulation, neutrophil and leukocyte chemotaxis, cellular response to lipopolysaccharide, cellular response to interferon-gamma, cellular response to interferon-beta, TNF signaling and TLR signaling (FIG. 6C). Consistent with the well-established links between dysregulated lipid metabolism and liver inflammation and damage, anti-CD40 treatment led to a significant down-regulation of genes involved in metabolism including lipid/fatty acid metabolism, steroid & cholesterol metabolism, amino acid metabolism, bile acid metabolism, and arachidonic acid metabolism.

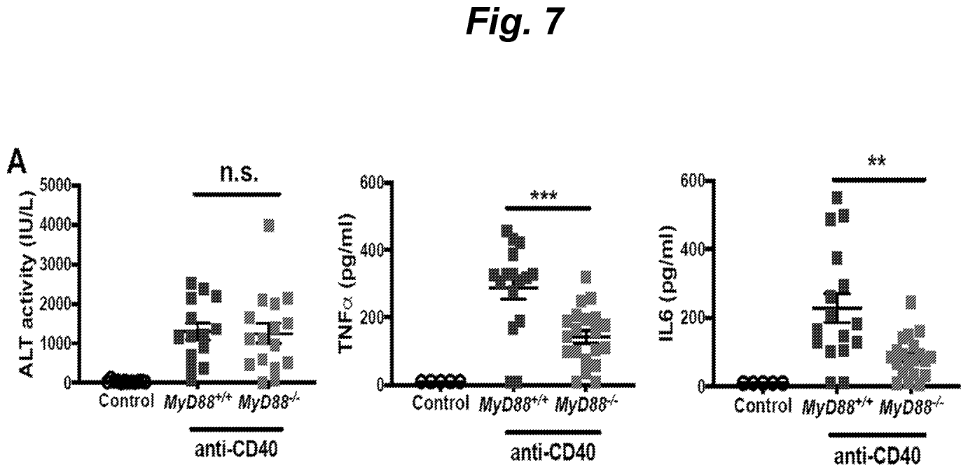

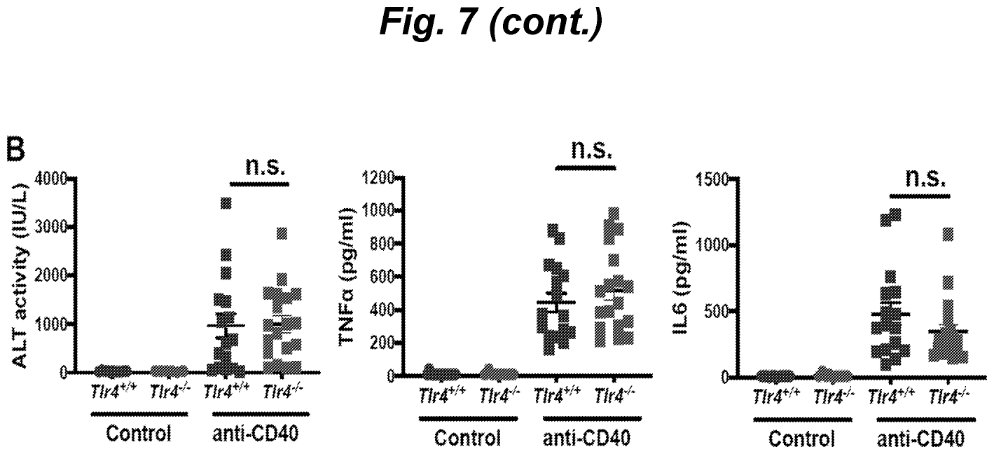

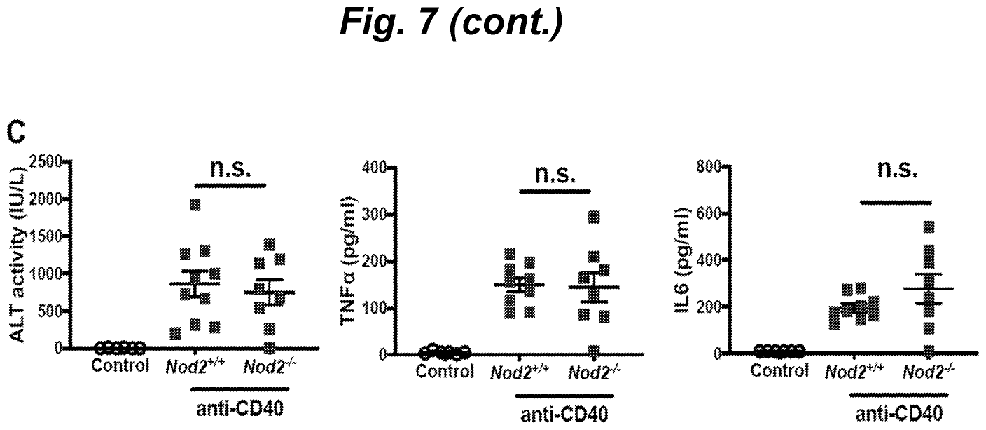

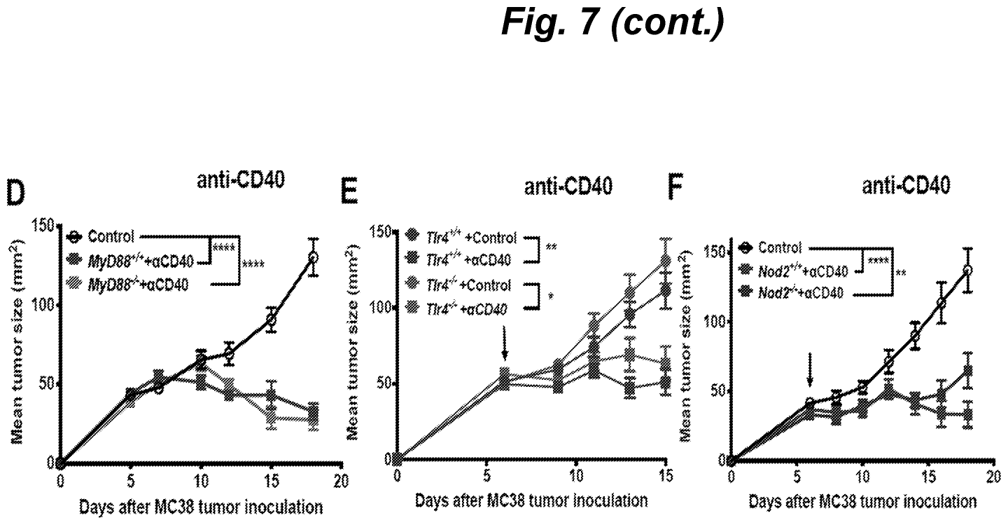

Concomitantly treating anti-CD40 treated mice with antibiotics significantly altered the expression of more than 500 genes compared to mice treated with anti-CD40 alone. Anti-CD40 treated ABX mice had significantly reduced expression of genes involved in the immune response, inflammation, chemotaxis and neutrophil degranulation, compared to mice treated with anti-CD40 alone (FIG. 7D,). Genes involved in chemotaxis that were down-regulated in ABX+anti-CD40 mice included Ccl3, Ccl4 (FIG. 7E, G-F) (also known as macrophage inflammatory protein (MIP)-1α and MIP-1β, respectively) and Ccr1 (encodes the receptor for MIP-1α), providing a plausible explanation for the significantly reduced infiltration of macrophages/monocytes observed in the livers of ABX mice following anti-CD40 treatment. MIP-1α and MIP-1α can also activate neutrophils and induce the synthesis of TNFα and IL6. Other key immune genes that were downregulated in ABX mice included Tlr4 and Il1β, as well as a host of other inflammatory response genes (FIG. 7F, H-I). In summary, anti-CD40 induces dramatic alterations in inflammatory and metabolic pathways in the liver which is dampened by treatment with antibiotics that presumably reduce activating signals from the gut microbiota.

Signaling Through MyD88 Modulates the CRS Induced by Anti-CD40 but Did not Alter Liver Damage or Anti-Tumor Efficacy.

Pattern recognition receptors such as the Toll-like receptors (TLRs) and nucleotide-binding oligomerization (NOD) like-receptors (NLR) recognize and bind specific microbial products from the gut microbiota such as LPS (TLR4) and peptidoglycan (NOD2). We hypothesized that the effects of the gut microbiota on anti-CD40 induced immunotoxicity could be mediated via microbial signals sensed by these pathways. This hypothesis was supported by liver RNAseq data that revealed that anti-CD40 treatment induced the up-regulation of genes involved in TLR and NLR pathways, many of which were dampened by antibiotic treatment. We therefore evaluated anti-CD40 induced toxicity in Myd88^{-/-} (an essential adaptor protein downstream of many TLRs), Tlr4^{-/-} and Nod2^{-/-} mice. Anti-CD40 induced serum ALT levels in Myd88^{-/-}, Tlr4^{-/-}, and Nod2^{-/-} mice were not significantly different to the wildtype control mice (FIG. 7A, B, C), suggesting that these pathways do not mediate the influence of the microbiota on anti-CD40 induced liver damage. Interestingly, MyD88^{-/-} mice did have significantly reduced anti-CD40 induced levels of TNFα and IL6 compared to wildtype mice (FIG. 7A). Levels of TNFα and IL6 induced by anti-CD40 were not significantly different in either Tlr4^{-/-} (FIG. 6B) or Nod2^{-/-} (FIG. 7C) mice, compared to wildtype mice. To further investigate why Myd88^{-/-} mice had significantly reduced anti-CD40 induced CRS compared to wildtype mice but comparable levels of serum ALT indicative of liver damage, we assessed liver immune cell infiltration in Myd88^{-/-} mice following anti-CD40 treatment. Apart from a modest reduction in neutrophil infiltration, there were no significant differences in the infiltration of NK cells, monocytes/macrophages, or cDCs into the livers of anti-CD40 treated Myd88^{-/-} mice compared to wildtype mice. We also investigated the anti-tumor efficacy of anti-CD40 in Myd88^{-/-}, Nod2^{-/-}, and Tlr4^{-/-} mice, which was not significantly different compared to matched wildtype mice (FIG. 7D-F). These data suggest that while a MyD88 dependent pathway mediates the CRS induced by anti-CD40, this pathway is not required for anti-CD40 induced liver damage or the anti-tumor efficacy of anti-CD40.

The Gut Microbiota Modulates Liver Bile Acids without Impacting Anti-CD40 Induced Liver Damage

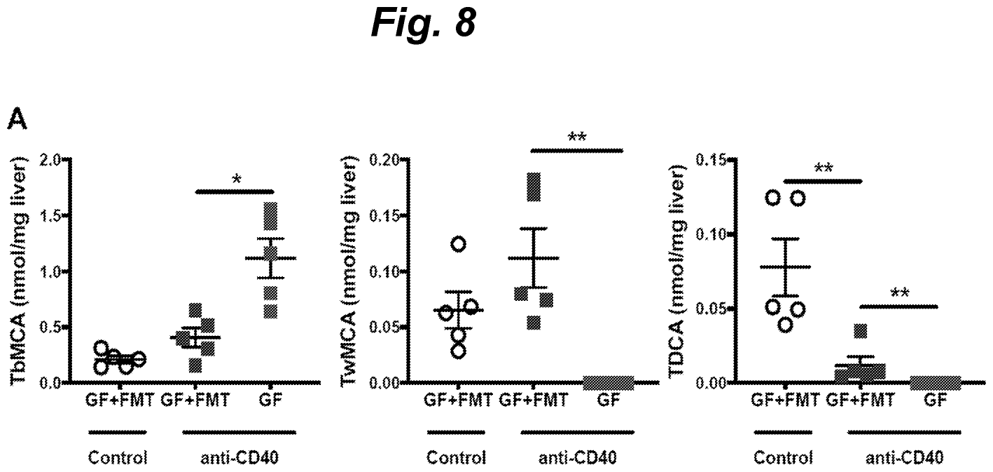

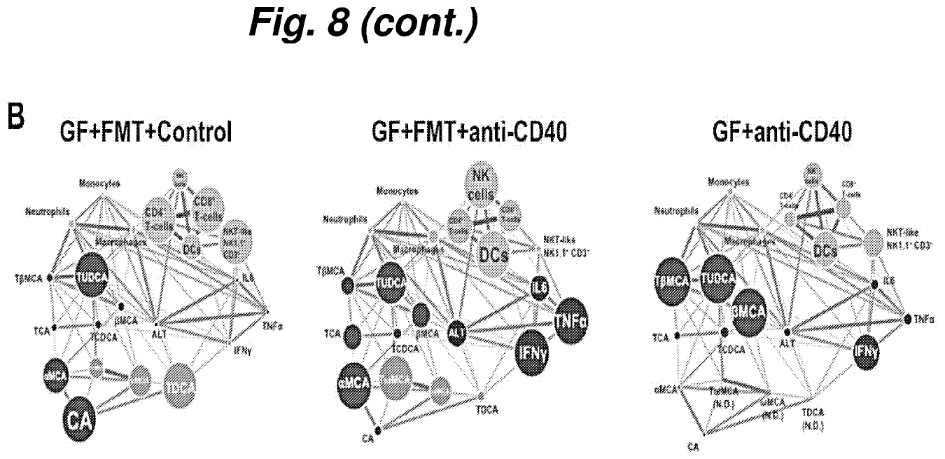

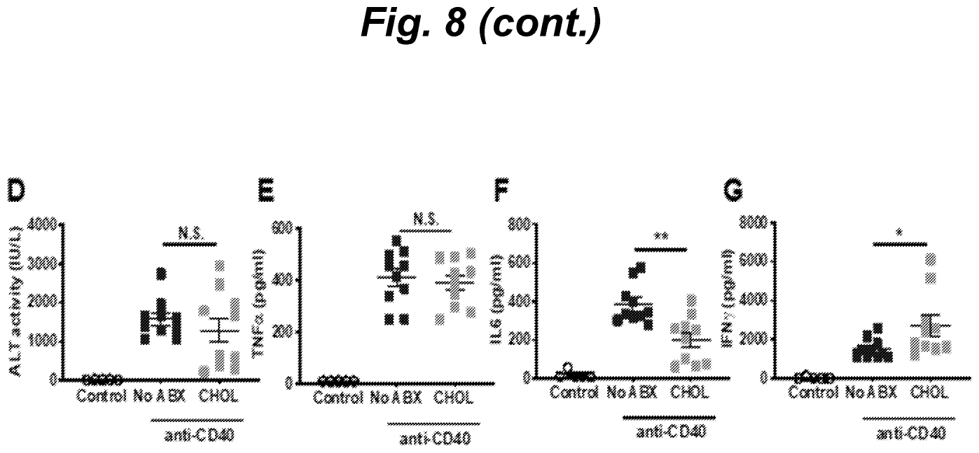

Bile acids play an important role in the crosstalk between the gut microbiota and immune responses in the liver and can potently modulate host immune and metabolic functions. Secondary bile acids are metabolized from primary bile acids by bacteria in the gut microbiota and can modulate the activity of NKT-cells and macrophages in the liver. RNAseq analysis also indicated that both anti-CD40 and antibiotic treatment led to significant alterations to the expression of genes involved in bile acid metabolism in the liver. To investigate this further, we quantified primary and secondary bile acids in the livers of anti-CD40 or control (PBS) treated SOPF, antibiotic treated, and germ-free (GF) mice, using targeted liquid chromatography mass spectrometry. Secondary bile acids (ωMCA, TDCA and TωMCA) were severely depleted in the livers of GF mice. (FIG. 8A) Consistent with previous reports, we also observed that the primary bile acid, TβMCA, was significantly increased in GF mice. Administering an FMT from normally colonized SOPF mice to GF mice led to significant increases in the levels of secondary bile acids and a corresponding reduction in TβMXA levels in the liver. We observed a similar, although weaker effect on bile acids in SOPF mice treated with antibiotics. Interestingly, the secondary bile acid, TDCA, was significantly decreased following anti-CD40 in both GF+FMT (FIG. 8A) and SOPF mice, suggesting that anti-CD40 treatment may be impacting the gut bacteria that produce TDCA

To further examines the potential associations between bile acid metabolism and anti-CD40 induced toxicity in the liver, we performed a network-based correlation analysis, representing the statistically significant (P<0.05) pairwise correlations between the levels of primary and secondary bile acids in the liver, the frequency of various immune cell populations in liver, and serum ALT and cytokine levels (which were all assessed in the same mice and were thus directly comparable). There were strong correlations between multiple different immune cell populations in the liver (macrophages, monocytes, neutrophils, cDCs and T-cells) and serum cytokine (TNFα, IL6, IFNγ) and ALT levels (FIG. 8B-C), suggesting that the CRS and liver damage induced by anti-CD40 is dependent on an anti-CD40 induced pathogenic immune cell infiltration into the liver. Clearly this immune cell infiltration is dependent on the gut microbiota as it is substantially reduced in antibiotic treated and GF mice and can be stored by FMT to GF mice. Interestingly, specific bile acids, such as TβMCA and TUDCA, were negatively correlated with the proportion of pathogenic immune cell populations (neutrophils and monocytes/macrophages) and with serum ALT

To investigate whether there was a causative relationship between bile acid levels and anti-CD40 induced liver damage and CRS, we depleted bile acids in mice by feeding them a 2% cholestyramine diet to sequester bile acids in the GI tract. As anticipated, cholestyramine had a global impact on liver bile acids, significantly reducing both primary and secondary bile acids. Depletion of liver bile acids with cholestyramine, however, did not reduce the liver damage induced by anti-CD40 as measured by serum ALT (FIG. 8D). Interestingly, the CRS induced by anti-CD40 was partially dependent on bile acid metabolism, as mice fed the cholestyramine diet had significantly reduced anti-CD40 induced levels of IL6 but not TNFα (FIG. 8E-F) and significantly increased levels of IFNγ (FIG. 8G). Given that anti-CD40 induced ALT levels were not significantly reduced in these mice, but IL6 levels were, these data also suggest that anti-CD40 induced liver damage is not mediated by IL6, but could be mediated by anti-CD40 induced TNFα, which was not suppressed in mice fed the cholestyramine diet.

Liver Damage Induced by Anti-CD40 is Driven by Macrophages, that Amplify Immune Inflammation Leading to Neutrophil Degranulation Through the Secretion of TNFα

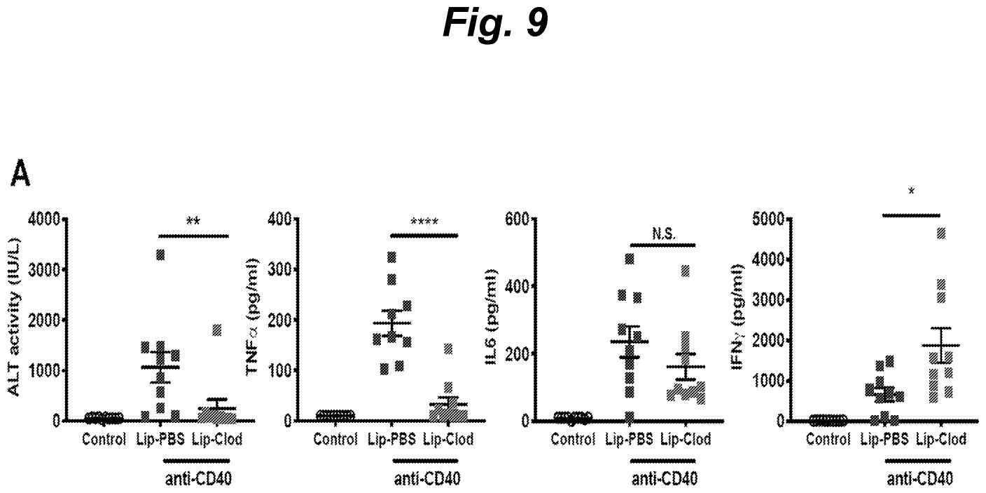

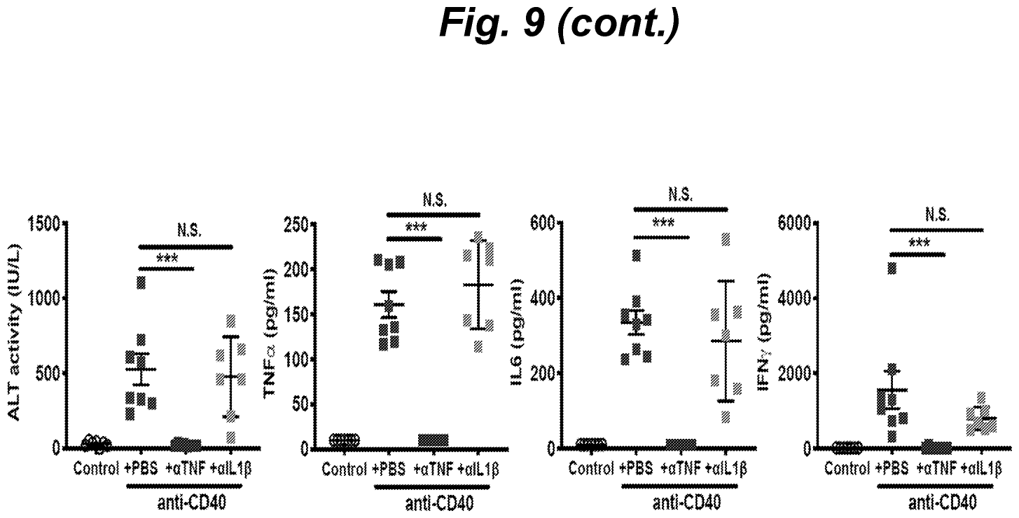

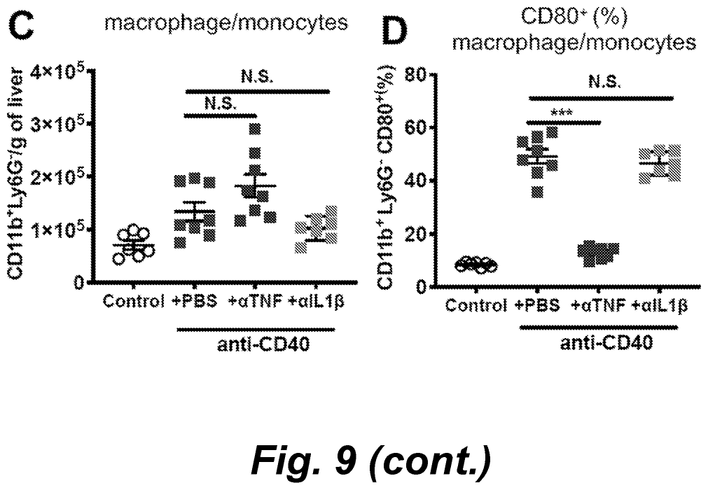

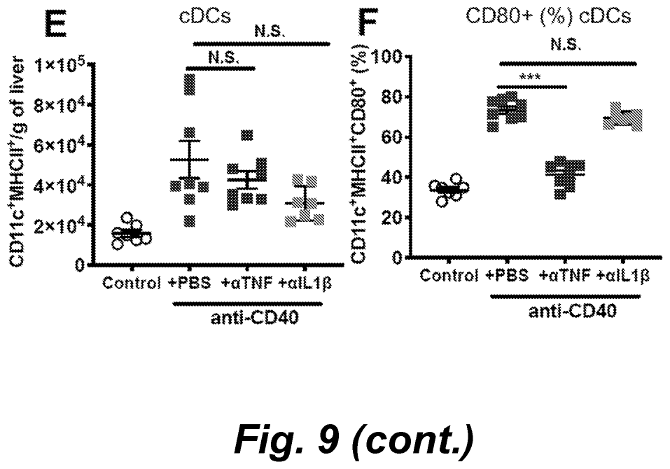

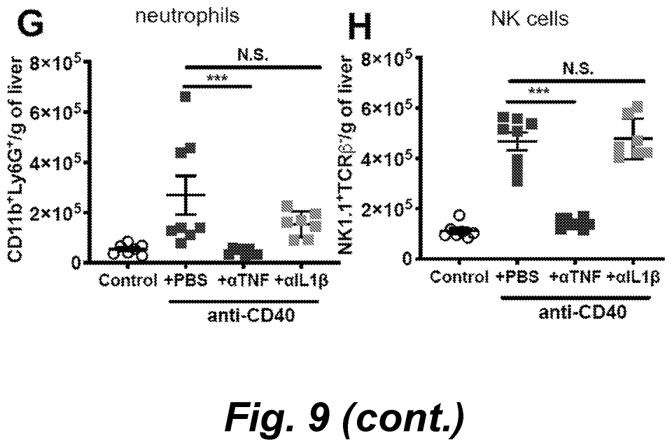

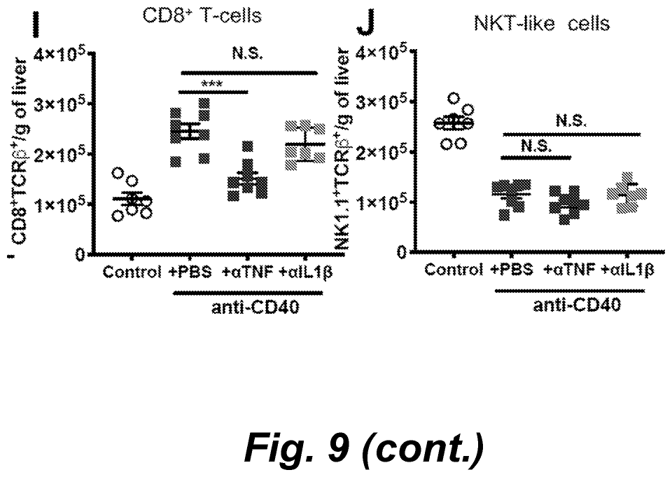

Given that macrophages are potent producers of TNFα and that both anti-CD40 induced macrophage infiltration into the liver and TNFα production are significantly reduced in antibiotic treated and GF mice, we hypothesized that the gut microbiota modulates the immunotoxicity induced by anti-CD40 by amplifying the macrophage response to anti-CD40 ligation, leading them to produce pathogenic levels of TNFα. To investigate this hypothesis, we evaluated the toxicity of anti-CD40 in mice depleted of macrophages by i.v. injection of clodronate liposomes 1 day prior to anti-CD40 administration Mice injected with clodronate loaded liposomes had ˜70% fewer liver macrophages (CD11b⁺Ly6G⁻F4/80⁻) compared to mice injected with control, PBS loaded liposomes. Correspondingly, mice depleted of their macrophages had significantly reduced levels of anti-CD40 induced TNFα and ALT in serum (FIG. 9A), indicating that these mice were protected against anti-CD40 liver damage in a TNFα dependent manner. Confirming this, mice treated with an anti-TNFα blocking antibody, but an anti-IL1β one, prior to anti-CD40 treatment, had completely abrogated anti-CD40 induced serum ALT levels (FIG. 9B). Interestingly, blocking TNFα did not alter anti-CD40 induced infiltration of macrophages or monocytes (FIG. 9C), rather it prevented their activation, almost completely blocking their upregulation of CD80 (FIG. 9D) and CD86. A similar effect on conventional dendritic cells was also observed (FIG. 9 E-F). Blocking TNFα also prevented the infiltration of neutrophils (FIG. 9G), NK cells (FIG. 9H) and CD8⁺ T-cells (FIG. 9I) the liver following anti-CD40 treatment, but did not alter the effect of anti-CD40 on NKT-like cells (FIG. 9J). These data demonstrate that anti-CD40 induces IL6 in a manner that is dependent on the gut microbiota, MyD88, and bile acid metabolism in the liver. Anti-CD40 induced liver damage is, on the other hand, mediated via TNFα and a pathogenic activation of macrophages into the liver, both of which are in turn dependent on co-activating signals from the gut microbiota.

Antibiotics can Significantly Reduce Liver Immune Inflammation Induced by Anti-OX40

A recent study evaluating the IAA anti-OX40 reported that OX40 was highly expressed on liver Natural Killer T (NKT) cells, and treatment with anti-OX40 initiated pyroptosis (inflammatory cell death) of liver NKT cells, inducing the secretion of inflammatory cytokines IL1β and IL18 causing damage to the liver Another recent study demonstrated that antibiotic (ABX) treatment increased liver NKT numbers and function by modulating the secretion of secondary bile acids from gut bacteria. These two studies suggested that the gut microbiome would also impact the toxicity of anti-OX40 immune agonist antibodies so we investigated if antibiotics could dampen anti-OX40 induced liver damage.

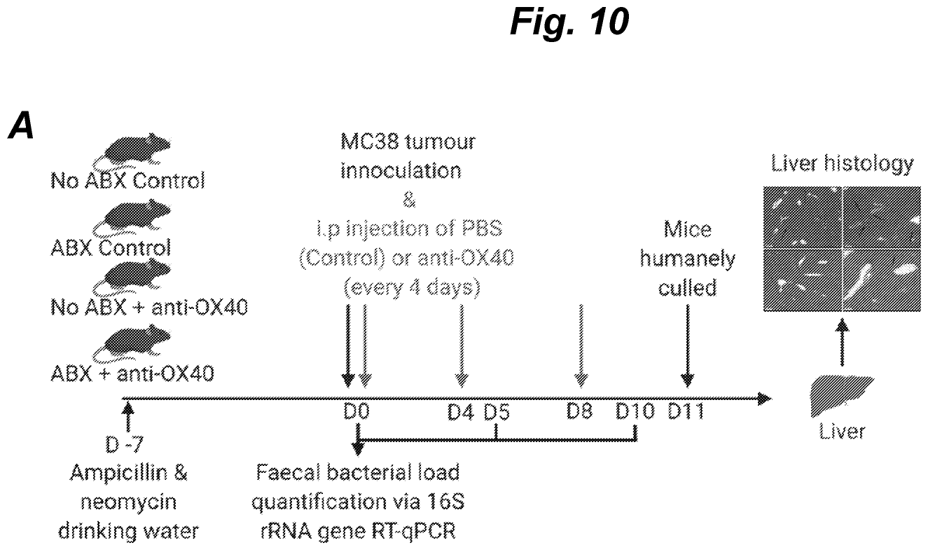

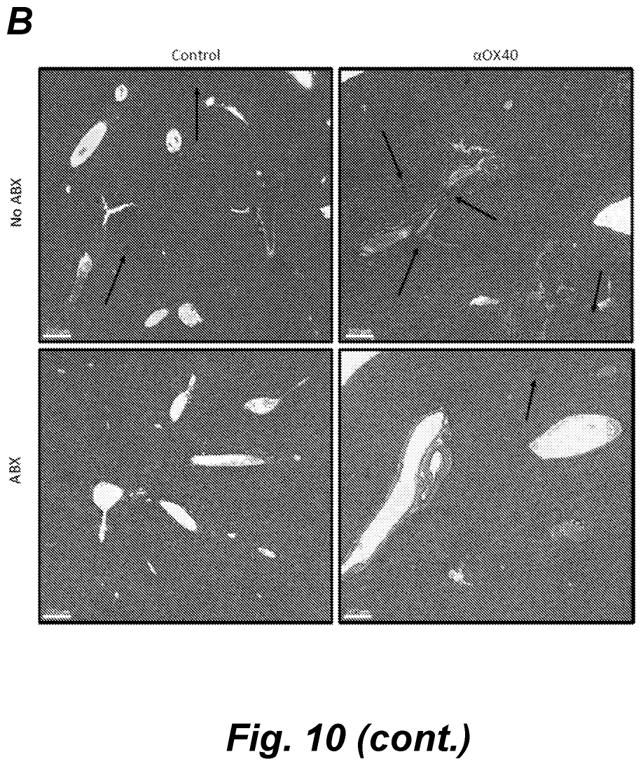

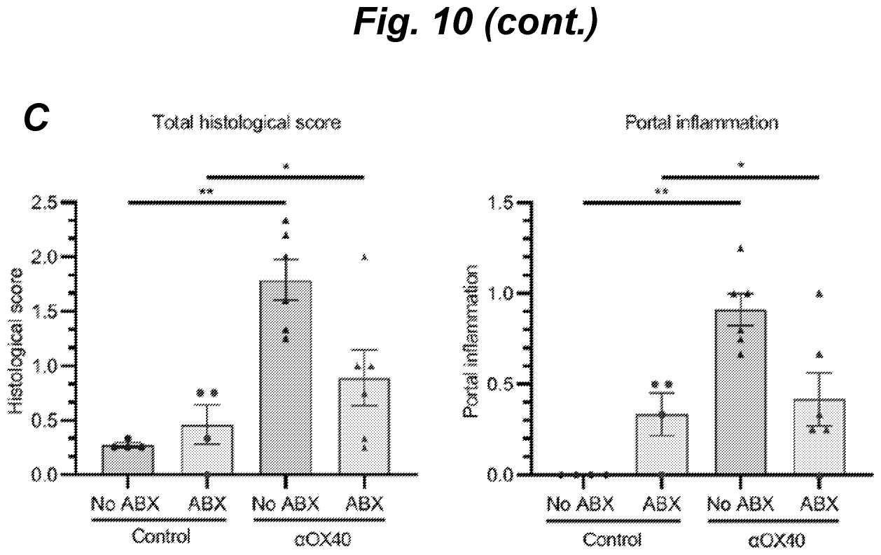

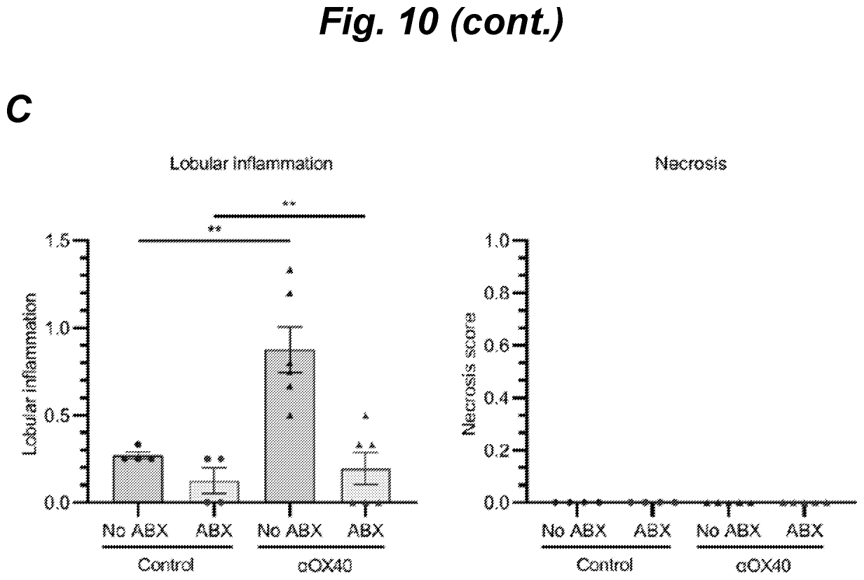

To investigate whether the toxicity and efficacy of αOX40 can be modulated by the gut microbiota, 4 groups of 5-week old, male C57BL/6 mice (n=10/group, (FIG. 10A) were treated with antibiotics to deplete their gut microbiota. 1 week later, tumors were then inoculated while mice were simultaneously treated with 100 μg of anti-OX40 or PBS vehicle control 3 times in 4 day intervals. To assess whether αOX40 could induce liver toxicity, formalin fixed sections of liver tissue were stained with hematoxylin and eosin (H&E) and were histologically scored for inflammation (FIG. 10B). Liver sections were scored based on portal inflammation (inflammation around the blood vessels), lobular inflammation (inflammation residing away from blood vessels and around hepatocytes) and necrosis (regions of hepatocyte death indicated by discoloration and lack of cell nuclei). The highest cumulative score is 9 with each type of inflammation contributing a maximum of 3 points. There was a significant increase in portal and lobular inflammation in the livers of anti-OX40 treated mice (FIG. 10C), but there was a lack of any visible necrotic regions and the histological score was modest indicating mild inflammation. Interestingly, we observed that ABX treatment significantly reduced the mild liver inflammation induced by αOX40 treatment. These data suggest that similar to what we have observed for other IAAs, the milder inflammation induced by αOX40, may also be partially modulated by the gut microbiota.

Discussion

In this study, we have identified the gut microbiota as a critical component in the induction of the immunotoxicity of anti-CD40 and anti-CD137 IAAs. Mice treated with antibiotics, or GF mice lacking a microbiota were protected from the liver damage, colitis and CRS induced by anti-CD40 and anti-CD137. In contrast to what has been observed with ICTs, we found that the gut microbiota had little impact on the anti-tumor efficacy of IAAs. These data demonstrate that, antibiotics, or other more targeted methods to inhibit the signals from the gut microbiota responsible, will improve the clinical safety of IAAs.

We found that the gut microbiota impact on IAAs toxicity was mediated via the alteration of liver immune populations, with GF and ABX treated mice having reduced levels of anti-CD40 driven infiltration and activation of macrophages and cDCs. We have also demonstrated macrophages are causative for the liver damage induced by anti-CD40 Interestingly, we also demonstrated that the gut microbiota modulates anti-CD137 driven macrophages/monocytes and CD8⁺ T-cell infiltration into the liver. We have also demonstrated the dependence of TNF, but not IL1β in the immunotoxicity of anti-CD40. Several recent studies also demonstrated TNFα blockade, but not IL6 also reduces the liver damage and colitis induced by anti-CD40. These studies are also supportive of our findings that IL6 is not required for anti-CD40 induced liver damage. However, we did identify that inhibiting bile acids or MyD88 signaling, both prominent pathways modulated by the gut microbiota, reduced IL6 secretion in response to anti-CD40. IL6 is a critical cytokine mediating the organ damage in a number of inflammatory conditions including sepsis, graft versus host disease and CRS induced by other immunotherapies such as blinatumomab or CAR-T cells. Therefore, there is potentially an important role of the gut microbiota signaling through bile acids and MyD88 in these conditions. In addition to the importance of TNFα, IL12 has recently been shown to be a critical cytokine in the immunotoxicity induced by anti-CD40. However, its ability to induce IL12 is critical for the anti-tumor activity of anti-CD40, particularly in combination with anti-PD1. We did not find IL12p40 secretion following anti-CD40 to be reduced by the gut microbiota (data not shown), nor an effect of ABX on the anti-tumor efficacy of anti-CD40, suggesting the microbiota effect on anti-CD40 toxicity to be independent of IL12.

While we have demonstrated the gut microbiota has a causative role in the immunotoxicity of anti-CD40 or anti-CD137, the pathways or species responsible for the full spectrum of toxicity remains to be determined. Mice deficient in MyD88 had a reduced anti-CD40 induced cytokine storm, with inflammatory cytokines TNFα and IL6 significantly reduced. However, despite demonstrating anti-CD40 toxicity is dependent on TNFα, liver damage and immune infiltration was similar between wildtype and Myd88^{-/-} mice, suggesting additional cytokines, or potentially TNFα production in liver macrophages may be the critical determinant in the toxicity of anti-CD40. There is also the possibility that the liver toxicity of anti-CD40 is dependent on signaling through both MyD88 and an additional pathway(s), supported by a recent study that found that triple deficiency in PRR adaptor molecules, MyD88, TRIF and CARDIF was required prevent the effect of the microbiota on priming DC activity. We also found MyD88 was not required for anti-CD40 anti-tumor activity. Other potential microbiota pathways contributing to the toxicity of IAAs may include short chain fatty acids, such as propionate, butyrate and acetate have been shown to modulate many different immune functions, including macrophage activation. Identification of the pathways/microbiota products responsible for increasing IAA toxicity will allow more targeted interventions, such as small molecule inhibitors of key bacterial genes or targeted antibiotics to deplete detrimental bacteria. Finally, there is the potential that some microbiota signals/species may dampen the toxicity of TAAs, allowing a probiotic approach to balance signals that increase IAA toxicity, with those that reduce their toxicity.

The outcome of this study has immense clinical relevance as there are approximately 20 different IAAs targeting 8 different co-activating receptors currently in phase I-III clinical trials, several of which induce serious dose limiting toxicities. Understanding and characterizing the role or the gut microbiota in IAA toxicity development may prove a useful prognostic tool to identify patients at greater risk of serious side effects. Indeed, the development of severe irAEs by IAAs is not universal, with serious grade 3 or above side effects observed in ˜30% anti-CD40, and ˜20-60% of anti-CD137 treated patients dependent on dose used. Patient microbiota sequencing prior to treatment with IAAs to determine associations with increased liver damage, CRS and colitis will be of great interest to confirm the translatability of our findings. Additionally, blocking the microbiota pathways responsible for increasing the toxicity of TAAs via antibiotics or more targeted approaches may make them safer for use in patients, potentially allowing higher more effective doses to be used. For example, urelumab is considered the most potent anti-CD137 agonist in clinical development, however phase I-II trials were halted due to high rates of dose dependent liver toxicity, resulting in several deaths. Clinical trials using an approximately 3-30 times lower dose of 8 mg/patient, alone or in combination with anti-PD1 have reported overall disappointing results. If we could control the liver toxicity of urelumab by targeting the gut microbiota, patients could potentially be safely treated with higher, more effective doses, improving responses to therapy. This is also highly relevant to several anti-CD40 IAAs, with dose limiting toxicities identified in early clinical trials meaning most subsequent studies have utilised CD40 IAAs at lower doses. Apart from an anti-CD28 IAA which induced severe CRS in all patients tested, other IAAs currently in clinical development; anti-CD27, anti-ICOS, anti-GITR and anti-OX40 have not reported dose limiting toxicities in clinical trials to date. However, this does not mean that they will not induce toxicity in patients, as ICIs targeting PD1/L1 and CTLA4 also do not have dose limiting toxicities, however, induce a range of immune related adverse events (irAEs), generally of low grade that were not evident until they entered more widespread clinical use. Therefore, determining the impact of the microbiota on additional clinically advanced IAAs is warranted, both clinically and in pre-clinical models. Therapies that induce weaker irAEs, such as ICIs, are often difficult to identify in wildtype mice. Further IAA pre-clinical testing may benefit from use of mouse models that enhance irAEs development, such as mice transiently depleted of T-regs, mice with a humanized immune system, or mice pre-disposed to develop autoimmunity, such as non-obese diabetic mice. Finally, as we have demonstrated that the microbiota modulates liver damage induced by IAAs by preventing immune infiltration/activation, it may also modulate the liver damage induced by other therapies such as ICIs. While uncommon when used as monotherapies, severe grade 3 and above liver damage occurs in ˜20% of anti-PD1/anti-CTLA4 combination treated patients.

The path to clinical translation of IAAs has been hampered by a number of roadblocks including their induction of severe toxicity, less than ideal efficacy as a monotherapy and competition from ICIs, which have received far more attention from researchers and biotech companies over the last decade. However, with second generation ICIs demonstrating activity in similar tumor types to those already targeted by existing anti-PD1/L1/CTLA4 inhibitors, there is a chance they will not be any more effective than anti-PD1 in tumors that are not robustly infiltrated by immune cells. This will lead to a major gap in treatment options for numerous immunologically cold tumors, however due to their potent systemic activity, IAAs are ideal therapies to drive tumor immune infiltration. Because of this activity, research into IAAs is experiencing a resurgence of interest over the last few years. Results from high-profile clinical trials testing anti-CD40 and anti-PD1 combination therapies in pancreatic cancers insensitive to almost all cancer treatments are due shorty and are eagerly awaited. The progression of the IAA, anti-ICOS, to phase 3 clinical trials and expansion of IAA testing into 70 clinical trials (clinicaltrials.gov) also underscores this resurgence of interest. Despite these advances, the toxicity associated with IAAs remains a concern. This study has demonstrated that the gut microbiota is a critical mediator of the toxicity of IAAs. These novel data raise the prospect of several new clinical interventions including approaches targeting signals from the microbiota to reduce toxicity and to enhance increase the dose of IAAs that can be safely administered. It may be also possible to use data on a patient's microbiota to predict risk of IAA-induced toxicity. Further investigation to identify which microbiota species and host pathways are driving the immunotoxicity of IAAs, as well as clinical studies to determine the translatability of these exciting results are strongly warranted.

Experimental Model and Subject Details

Mice: Specific and Opportunistic Pathogen Free (SOPF) C57BL/6 mice were bred and maintained under standardized conditions with regulated temperature, humidity and daylight at the South Australian Health and Medical Research Institute (SAHMRI). Colony founders were sourced from the Jackson Laboratory. Littermate wildtype and Tlr4^{-/-} and Myd88^{-/-} mice were generated by backcrossing to C57BL/6 mice. Nod2^{-/-} mice were co-housed with wildtype C57BL/6 mice for 4 weeks prior to use in experiments. The genotype of the mice was confirmed by a commercial provider (Garvan Institute) using PCR protocols provided by Jackson Laboratories.

Gnotobiotic (GF) C57BL/6 mice were purchased from the Translational Research Institute, Brisbane and housed at SAHMRI in positively pressurized, high-efficiency particulate air-filtered isolators (Park Biosciences) or HEPA filtered ISO-P cages (TECNIPLAST), with regulated temperature, humidity and daylight. GF mice had constant access to autoclaved commercially pelleted food and sterilized water and were tested for sterility regularly, with water, feces and bedding tested commercially (ComPath) or by assessing bacterial load in feces by 16S rRNA gene RT-qPCR. Both male and female mice were used except for mice inoculated with AT-3 breast cancer cells, where only female mice were used. All experiments and procedures were executed in accordance with protocols approved by the SAHMRI Animal Ethics Committee.

Antibiotic treatment: Mice were treated with neomycin (0.5 mg/ml) and ampicillin (1 mg/ml) dissolved in sterile drinking water. Mice had free access to treated drinking water throughout the experiments and the water was replaced three times weekly. Control mice had access to untreated drinking water ad libitum.

Fecal microbiota transplant into GF mice: To establish an intestinal microbiota, gnotobiotic mice received fecal microbiota transplants from SOPF mice. The cecal contents of aged-matched healthy, untreated control mice were extracted under anaerobic conditions. Cecal contents were pooled and diluted 3-fold in anaerobic (PBS). Gnotobiotic mice were administered the cecal material suspension via oral gavage, with a sterile flexible canicular. Successful microbiota colonization was confirmed by 16S rRNA gene qPCR or 16S rRNA gene sequencing.

Bacterial culture and transplant into GF mice: Enterobacter cloacae was isolated from the cecal contents of a SOPF C57BL/6 mouse at SAHMRI Bioresources that was colonized with high levels of Enterobacter (as shown by 16S rRNA gene sequencing). Cecal contents were collected in PBS, homogenized into solution and serial dilutions were plated out under anaerobic conditions. Single colonies that were identified as Enterobacter cloacae using a MALDI Biotyper (Bruker Daltonik), were streak plated to obtain a pure culture. Whole genome sequencing was performed on DNA extracts for bacterial strain confirmation based on the Kraken tool. 81% of the reads were mapped to the complete genome of Enterobacter cloacae.

Akkermansia muciniphila (DSM 22959) and Clostridium scindens (DSM 5676) were purchased commercially (DSMZ, Germany). Monocultures were prepared from a pure culture plate under aerobic conditions using brain and heart infusion broth at 37° C. Briefly. 200 mL BHI broth or BHI with mucin (5%) was inoculated with 20 mL of the overnight culture and grown to an OD₆₀₀ of 0.9. Pellets were produced by spinning down bacteria and washed in 1×PBS and resuspended in 1×PBS at 1×10⁹ CFU/mL. 100 μl of the suspension was administered to GF mice via oral gavage. Oral gavage was repeated 2-3 times, every 2-6 days and the monocultures were allowed to establish for 3 weeks prior to tumor inoculation.

Macrophage depletion: To deplete macrophages, mice were injected intravenously with 10 μL/g of bodyweight with either PBS, or clodronate loaded liposomes (5 mg/ml) as per manufacturer's instructions (Clodronateliposomes.org, The Netherlands). Depletion was performed 24 hours prior to administration of immune agonists.

Cell line culture: The MC38 cell line, derived from C57BL/6 murine colon adenocarcinoma cells (Corbett et al. (1975) Cancer Res 35: 2434-2439), was kindly donated by Dr. Susan Woods (SAHMRI). The AT-3 breast cancer line, established from cells of the primary mammary gland carcinoma of a MTAG transgenic mouse (Stewart and Abrams (2007) J Immunol 179: 2851-2859), was kindly donated by Fernando Souza-Fonseca-Guimaraes, (Walter and Elisa Hall Institute). Cell lines were cultured in culture medium (Dulbecco's modified eagle medium, 10% Fetal bovine serum, 2 mM glutamine, 1 mM Sodium Pyruvate and Penicillin-Streptomycin), at 37° C. 10% CO₂ and were routinely tested for mycoplasma contamination using the MycoAlert™ assay (Lonza, USA). Cell lines were also confirmed to be free of 13 common mouse pathogens by commercial PCR testing (Compath). Cell line authentication was not performed. Cell lines were passaged 3 times weekly in log phase by detaching adherent cells using trypsin-EDTA, centrifuging at 350 G and reseeded in flasks at a 1:5-1:10 dilution.

Tumor inoculation and monitoring: Cell lines in log phase at 60-85% confluency were detached by trypsin-EDTA, washed once and resuspended to 1×10⁷ cells/ml in DMEM without any additives. 1×10⁶ cells (100 μL) were injected subcutaneously into the flank of mice. Female and male mice were injected with MC38 tumors while the AT-3 breast cancer cell line was injected into female mice only Tumor growth was measured with Vernier Calipers weekly until the tumor area reached >50 mm² after which tumor growth was measured 3 times weekly. Area was calculated as width×breadth. Mice were humanely euthanized through CO₂ exposure when they reached a maximum tumor size of 1000 mm³.

Antibody administration: Mice were injected intraperitoneally (i.p.) with 100 μg of anti-CD40: clone FGK4.5 (Isotype, Rat IgG2a); anti-CD137 clone 3H3 (Isotype: Rat IgG2a), anti-OX49: clone OX86 (Isotype: Rat IgG2a),Rat IgG2a isotype control, clone 2A3; or 200 μg of anti-PD1: clone RMP1.14 (Isotype Rat IgG2a); anti-TNF: clone XT3.11 (Isotype Rat 1gG1); anti-IL1 β: clone B122 (Isotype Armenian hamster IgG) that were all purchased commercially (Bio X Cell) and diluted in a solution of PBS for injection. Control mice were injected with either PBS vehicle control or Rat IgG2a isotype control. Neither PBS nor isotype control IgG2a induced serum ALT, cytokines or impacted tumor growth. Mice were treated as indicated in figure legends.

Fecal sampling: Fecal samples were collected from mice at various time-points. Fecal samples were aseptically collected from individual mice and immediately snap frozen on dry ice and stored at −80° C. until processing.

DNA extraction from fecal samples: Fecal samples were individually weighed and resuspended in 1 mL of PBS, vortexed into a uniform suspension then pelleted by centrifugation at 13,000 rpm for 10 minutes. DNA was extracted from the fecal pellets using the DNeasy PowerLyzer PowerSoil Kit (Qiagen). The manufacturer's protocol was followed with the following modifications; fecal pellets were resuspended in 750 μL of Powersoil bead solution and 60 μL of Cl solution. The samples were incubated at 65° C. for 10 minutes prior to bead beating.

Quantitative real-time PCR analysis to determine bacterial load: Quantitative real-time PCR (RT-qPCR) was undertaken to assess bacterial load in collected fecal samples. RT-qPCR was performed on a Quant Studio™ 7 Flex Real-time PCR system using primers outlined in key resource table that target the conserved region of the 16S rRNA gene. Each reaction consisted of 5 μL SYBR green, 0.2 μM of each forward and reverse primer and 3 μL DNA template diluted in sterile water. Reactions were performed in triplicate and run against a no template control containing sterile water, substituted for the DNA template. The amplification program was 50° C. for 2 minutes, 95° C. for 10 minutes, 40 cycles of 95° C. for 15 seconds and 60° C. for 60 seconds. The total bacterial load was quantified against a standard curve of serial dilutions of E. coli genomic DNA.

16S rRNA gene sequencing: DNA extracted from fecal pellets were used to generate amplicons of the V4 hypervariable region of the 16S rRNA gene as described previously (Lynn et al. (2018) Cell Host Microbe 23: 653-660 e655). Sequencing of the amplicon library was performed using an Illumina Miseq system (2×300 bp run). 16S rRNA library preparation and sequencing was performed by the SAHMRI Genomics core. Paired-end 16S rRNA gene sequences were demultiplexed and imported into QIIME2 (release 2019.9) for processing. Sequences were error corrected, and counts of error-corrected reads per sample, which we refer to herein as exact sequence variants (ESVs), were generated with DADA2 version 1.8. A phylogenetic tree of error-corrected sequences was constructed with FastTree. Taxonomy was assigned to sequences with the sklearn plugin for QIIME2 with an 80% confidence threshold, using the GreenGenes 13.8 database. Further statistical analysis was carried out in R version 3.6.3, with graphing performed using ggplot2. Alpha and Beta diversity values were generated using PhyloSeq version 1.3.

16S rRNA SANGER sequencing for identification of novel bacterial strains: Sanger sequencing to identify bacterial monocultures was undertaken using a protocol described by the University of Pennsylvania School of Veterinary Medicine, Center for Host-Microbial Interactions. Briefly, PCR amplification of DNA extracted from fecal samples using a reaction consisting of NEBNext High-Fidelity PCR Master Mix, 0.2 μM 27F primer, 0.2 μM 1492R primer and DNA template between 150-250 ng/μL. The amplification program was set at 95° C. for 2 minutes. 30 cycles of 95° C. for 30 seconds, 55° C. for 30 seconds and 72° C. for 1 minute 40 seconds, and 1 cycle of 72° C. for 5 minutes. Samples were then incubated for 5 minutes with Sera-Mag™ Select DNA beads and placed on a magnetic stand until beads aggregated, and the solution was clear. Beads were then washed twice with 80% ethanol by incubating on a magnetic stand for 30 seconds and supernatant was discarded. Samples were then removed from the magnetic stand, resuspended in PCR grade water and incubated for 2 minutes, then placed back on a magnetic stand until the liquid was clear. Supernatant was then collected and Qubit quantification was performed on samples using the Qubit™ dsDNA BR Assay Kit following manufactures instructions. DNA concentrations was then adjusted to 25 ng/μL with PCR grade water Samples were sequenced by Australian Genome Research Facility, with each reaction consisted of sample at 25 ng/μL, 3 μM 515F primer and PCR grade water. Sequences were analyzed using Seq Scanner 2 to confirm monoculture purity. Chromatograms were visually inspected to confirm that a single unique product was sequenced. The species identify was assigned via a BLASTn search against the NCBI nr nucleotide database.

Liver RNA extraction: Liver sections (50-100 mg) were collected, snap frozen and stored at −80° C. Frozen liver samples were ground into powder and resuspended in TRIzol and incubated at room temperature for 15 minutes. The liver suspension was centrifuged for 2 minutes at 12,000 g and supernatant collected to eliminate particulate matter RNA was extracted following the manufacturer's protocol. Genomic DNA was removed from the RNA elution with the DNA-Free™ DNA Removal Kit (Thermofisher) following the manufacturer's instructions. Following DNase treatment, purity of RNA was improved by performing a sodium acetate re-precipitation by adding 1/10 dilution of 3M sodium acetate and 100% ethanol to reach a 70% ethanol concentration. Samples were then incubated overnight at 20° C. Samples were then spun for 30 min at 12000 g, washed twice in ethanol, allowed to air dry, and resuspended in RNase free water. RNA was quantitated using the QuBit RNA Broad Range Assay Kit (Qiagen) and RNA integrity was confirmed using a Bioanalyser (ThermoFisher).

RNA Sequencing: Library preparation and RNA sequencing of liver RNA was undertaken by the SAHMRI Genomics core facility. Briefly, total RNA was converted to strand specific Illumina compatible sequencing libraries using the Nugen Universal Plus mRNA mRNA-Seq library kit from Tecan (Mannedorf, Switzerland) as per the manufacturer's instructions (MO1442 v2). Briefly, 500 ng of total RNA was polyA selected and the mRNA fragmented prior to reverse transcription and second strand cDNA synthesis using dUTP. The resultant cDNA was end repaired before the ligation of Illumina-compatible barcoded sequencing adapters. The cDNA libraries were strand selected and PCR amplified for 12 cycles prior to assessment using an Agilent Tapestation to assess quality and a Qubit fluorescence assay for quantification. Sequencing pools were generated by mixing equimolar amounts of compatible sample libraries based on the Qubit measurements. Sequencing of the library pool was done using an Illumina Novaseq 6000 using a Si flowcell with 2×100 bp paired-end reads.

Fastq read quality was visualized with FastQC version v0.11.3 and summarized with MultiQC. Adapter sequences were removed with Cutadapt v2.8, and remaining sequences were quality filtered with Trimmomatic v0.38 where a sliding window with a minimum PHRED score of 20 with a window size of 4, together with average quality of 30. Ribosomal RNA levels were estimated with SortMeRNA v2.1. Reads were then aligned to GRCm38 mouse genome (Ensembl release 99 annotation) with HiSAT2 v2.1.0 on default alignment parameters. Feature Counts v1.5.0-p2 was used to count aligned reads. Per sample counts were then imported into R v3.6.3 for further statistical analysis. Gene sets were filtered for at least 1 count per million in 3 samples prior to analysis and unknown sources of variation were removed using SVAseq. EdgeR was used to normalize the data (using trimmed mean of M-values method) and perform differential expression analyses (with the glmLRT function). Pathways and Gene Ontology (GO) enrichment analysis was performed using a hypergeometric test implemented in R version 3.6.

Data availability: 16S rRNA gene sequence data have been deposited in the NCBI Sequence Read Archive under BioProject accession number PRJNA668656. RNA-Seq data have been deposited in the Gene Expression Omnibus (GEO) under accession number GSE15976.

Serum alanine aminotransferase (ALT) assay: Serum ALT levels were measured using the Liquid ALT Reagent Set (Pointe Scientific) following the manufacturer's instructions with the following modification. The reaction was scaled down from a 1 ml volume to 200 μL, to allow multiple samples to be run on a 96-well clear bottom black sided plate ALT levels were measured on an EnSpire® Multimode Plate Reader (PerkinElmer) spectrophotometer at a temperature of 37° C.

Cytokine ELISA: Serum IFN-γ levels were measured using the Mouse IFN-γ enzyme-linked immunosorbent assay (ELISA) kit (BD Biosciences). Serum TNF-α levels were measured using the Mouse TNF-α Uncoated ELISA Kit (Invitrogen). Serum IL-6 were measured using the Mouse IL-6 Uncoated ELISA Kit (Invitrogen). All ELISAs were run in accordance with the manufacturer's specifications

Flow cytometry: Tumors and livers were collected for immunophenotyping by flow cytometry. Single-cell suspensions were generated by mincing tumor tissue with scissors and incubating with 1 mg/ml Collagenase type IV, 500 ng/ml DNase I and 2% FBS in RPMI 1640 for 45 minutes at 37° C. Tumor samples were then pushed through a 40 μm cell strainer and washed with PBS. Livers were minced with scissors and pushed through a 70 μm cell strainer. Leukocytes were selected by resuspending cells in a single layer of 37.5% Percoll and centrifuging samples for 12 minutes at 690 g, supernatant was discarded. Ammonium-Chloride-Potassium lysis buffer was added to the pellet to lyse erythrocytes Liver/tumor samples were resuspended in FC blocking antibody Clone 2.4G2 (anti-CD16/32) in FACS buffer (PBS, 0.1% BSA 2 mM EDTA) in order to block Fc receptors and stained with the antibodies outlined in the key resource table (all from BD Biosciences, eBioscience, Miltenyi or Biolegend) and incubated for 30 minutes on ice. In some experiments, intracellular staining was performed to detect transcription factors in cells. Samples were washed in FACS buffer and fixed in FoxP3 staining buffer (eBioscience) for 30 minutes on ice. Samples were then washed with perm buffer and intracellular antibodies; FoxP3 and Ki67 (BD Bioscience) were added to samples and incubated for 30 minutes on ice; samples were washed with perm buffer followed by FACS buffer. Cells were then resuspended in FACS buffer and run on the BD LSRFortessa™ X-20 (BD bioscience). Dead cells were stained with DNA binding dyes DAPI (BD Bioscience) or ZombieAqua (Biolegend) prior to running on a flow cytometer and were excluded from analysis. Samples were washed in FACS buffer, and cells were resuspended in FACS buffer and liquid counting count beads (BD Biosciences) were added to samples to determine absolute counts. Samples were run on the BD LSRFortessa™ X-20 (BD bioscience). Data was analyzed using FlowJo™ v10.6.1 (FlowJo).

Isolation and profiling of bile acids from mouse liver: Snap frozen liver segments (˜50-100 mg) were spiked with a known concentration of each internal standard (D4-chenodeoxycholic acid, D4-cholic acid, D4-deoxycholic acid, D4-glycodeoxycholic acid, D4-glycochenodeoxycholic acid and norcholic acid) and then 500 μL of 0.2 M NaOH were added. After homogenization in a bead beater using zirconia/silica beads (1 mm diameter, Daintree Scientific), the liver homogenates were incubated for 20 minutes at room temperature. Fats were removed through liquid:liquid extraction using hexane and bile acids isolated from the aqueous phase through solid phase extraction using OASIS-HLB columns (Waters) as previously described (Sayin et al. (2013) Cell Metab 17: 225-235.). Bile acids were eluted in 90% acetonitrile:water, dried using a nitrogen stream and reconstitute in 1:1 methanol:water. Chromatographic separation and bile acid identification were achieved through liquid chromatography-mass spectrometry as previously described Caparros-Martine et. Al. (2017 Microbiome 5: 95.

Organ histological analysis: Portions of the liver were fixed in 10% neutral buffered formalin for 7 days and transferred to an 80% ethanol solution for storage. Liver sections were embedded in a paraffin block and lateral cross-sections were cut and stained with hematoxylin and eosin (Histology Services, University of Adelaide). Slides were then scanned using a SCN400 F Brightfield and Fluorescence Slide Scanner (Leica Microsystems) at 20× magnification and the CaseViewer software (3DHISTECH Ltd) was used to visualize samples. Liver, skin and lung sections were histologically scored in accordance with the protocol described in Mayer et al. (2014) Eur J Immunol 44: 2990-3002.

Statistical analysis: Data was analyzed using a Mann-Whitney test in GraphPad Prism 8 (GraphPad Software Inc.) or as otherwise stated in the figure legends. P-values ≤0.05 were considered statistically significant.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise. Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date

1. A method of reducing one or more side effects associated with immunotherapy using an immune agonist antibody in a subject suffering from a cancer, the method comprising modifying the gut microbiota in the subject and thereby reducing the one or more side effects associated with the immunotherapy in the subject.

2. The method according to claim 1, wherein the immune agonist antibody comprises one or more of an anti-CD137 antibody, an anti-CD40 antibody, an anti-CD28 antibody, an anti-GITR antibody, anti-OX40 antibody, an anti-CD27 antibody, and an anti-ICOS antibody.

3. The method according to claim 1, wherein the immunotherapy further comprises use of an immune checkpoint inhibitor.

4. The method according to claim 3, wherein the immune checkpoint inhibitor comprises one or more of an anti-PD1 antibody, an anti-CTLA4 antibody, an anti-PDL1 antibody, an anti-TIGIT antibody, an anti-Lag3 antibody, and an anti-Tim3 antibody.

5. The method according to claim 1, wherein the one or more side effects comprises one or more of liver toxicity, increased cytokine expression, cytokine release syndrome, colitis, dermatitis, elevated levels of liver enzymes in serum, liver necrosis, a haematological toxicity, and death.

6. The method according to claim 1, wherein the modifying the gut microbiota comprises one or more of administration to the subject of an antibiotic, administration to the subject of probiotic bacteria, a faecal transplant, administration to the subject of a prebiotic, administration to the subject of a probiotic with a prebiotic, administration to the subject of one or more suitable foods and/or supplements, and administration to the subject of bacteriophage.

7. The method according to claim 1, wherein the method does not substantially decrease efficacy of the immunotherapy.

8-10. (canceled)

11. A method of preventing and/or treating one or more side effects associated with treatment of a subject with an immune agonist antibody, the method comprising modifying the gut microbiota of the subject prior to, concurrently with, and/or following treatment with the immune agonist antibody.

12. The method according to claim 11, wherein the method comprises assessing the susceptibility of the subject to the one or more side effects associated with the immune agonist antibody immunotherapy by assessing the abundance of one or more specific species or strains of bacteria in the gut microbiota of the subject.

13. A method of increasing the dose of an immune agonist antibody administered to a subject for immunotherapy by reducing one or more side effects associated with the administration of the immune agonist antibody, the method comprising modifying the gut microbiota in the subject and thereby increasing the dose of the immune agonist antibody able to be administered to the subject.

14-18. (canceled)

19. A method of treating a subject suffering from a cancer with an immune agonist antibody, the method comprising
treating the subject with the immune agonist antibody; and
modifying the gut microbiota of the subject prior to, concurrently with, and/or following treatment with the immune agonist antibody.

20. The method according to claim 19, wherein the modifying of the gut microbiota comprises one or more of administration to the subject of an antibiotic, administration to the subject of probiotic bacteria, a faecal transplant in the subject, administration to the subject of a prebiotic, administration to the subject of one or more suitable foods and/or supplements, and administration to the subject of bacteriophage.

21. The method according to claim 19, wherein the method comprises assessing the abundance of one or more specific species or strains of bacteria in the gut microbiota of the subject.

22. The method according to claim 19, wherein the method is used to reduce one or more side effects in the subject associated with the treatment with the immune agonist antibody.

23-26. (canceled)

27. A combination product for treating a subject with an immune agonist antibody, the product comprising the following components:
an immune agonist antibody; and
one or more bacteria capable of modifying the gut microbiota and/or an agent capable of modifying the gut microbiota.

28. The combination product according to claim 27, wherein the immune agonist antibody comprises one or more of an anti-CD137 antibody, an anti-CD40 antibody, an anti-CD28 antibody, an anti-GITR antibody, an anti-OX40 antibody, an anti-CD27 antibody, and an anti-ICOS antibody.

29. The combination product according to claim 27, wherein the agent comprises an antibiotic, a prebiotic, one or more suitable foods and/or supplements, and/or bacteriophage.

30-35. (canceled)

36. The method according to claim 19, wherein the subject is treated with the immune agonist antibody alone, or in combination with an immune checkpoint inhibitor.

37. The method according to claim 36, wherein the immune checkpoint inhibitor comprises one or more of an anti-PD1 antibody, an anti-CTLA4 antibody, an anti-PDL1 antibody, an anti-TIGIT antibody, an anti-Lag3 antibody, and an anti-Tim3 antibody.
